(19) United States
(12) Patent Application Publication
(10) Pub. No.: US 2017/0028820 A1
(43) Pub. Date: Feb. 2, 2017

(54) AIR TREATMENT SYSTEMS FOR TRANSPORTATION ENCLOSURES AND RELATED METHODS

(71) Applicant: Clean Air For The People Inc., Marco Island, FL (US)

(72) Inventor: John WALSH, Marco Island, FL (US)

(73) Assignee: Clean Air For The People Inc., Marco Island, FL (US)

(21) Appl. No.: 15/294,878

(22) Filed: Oct. 17, 2016

Related U.S. Application Data
(63) Continuation of application No. PCT/US2015/026434, filed on Apr. 17, 2015.
(60) Provisional application No. 61/980,977, filed on Apr. 17, 2014.

(51) Int. Cl. B60H 3/06; A61L 9/20

(52) U.S. Cl. CPC B60H 3/0608; A61L 9/20; A61L 2209/14; B60H 2003/0675

(57) ABSTRACT

The present application provides for air treatment systems for treating air input into a transportation enclosure via at least one input. The air treatment systems include at least two filters that increase in ASHRAE 52.2 Standard MERV parameters in the direction of the flow of air. The filter positioned furthest downstream is at least a parameter 8 MERV ASHRAE Standard 52.2 filter. The air treatment systems include at least one ultraviolet (UV) light emitting device, and UV light reflective material. UV light emitted from the at least one UV light emitting device irradiates at least a portion of the plurality of filters. The plurality of filters, at least one ultraviolet (UV) light emitting device, and UV light reflective material provide a dose of UV light of at least 20 J/m² to the air input into the transportation enclosure via the at least one input.

### CROSS-REFERENCE TO RELATED APPLICATION

This application is a continuation of International Application No. PCT/US2015/026434, filed Apr. 17, 2015, which claims the benefit of U.S. Provisional Application No. 61/980,977, filed Apr. 17, 2014, which are hereby incorporated herein by reference in their entirety.

### BACKGROUND

The present disclosure generally relates to air treatment systems, and more particularly to air treatment systems for heating, ventilation and air conditioning systems of transportation enclosures.

A large percentage of the world population is dependent upon forms of public and private transit systems. A significant amount of employment absenteeism can be attributed to illnesses that have been contracted while within a transportation enclosure. Some experts have estimated that the economic impact from such transportation related absenteeism is in the billions of dollars per day just in the United States alone. Currently, data analysis is being conducted to accurately assess the actual economic impacts of transportation related illnesses and absenteeism. For example, research is being conducted at two major transit agencies in conjunction with MIT, the Harvard School of Public Health and various committees of the Transportation Research Board.

It was noted in the Clean Air Act of 1990 that exterior air quality was unacceptable as a result of vehicle systems (engines). The combination of engineering development and application of existing technology made exterior air quality from mass transportation vehicles acceptable. However, interior air equality of mass transportation enclosures has not been adequately addressed.

Research, when reviewed in conjunction with data produced by the CDC, the Dept. of Homeland Security, the U.S. EPA, St. Vincent's Hospital, the Johns Hopkins School of Public Health, the World Health Organization, Penn State University and numerous other research and scientific bodies, unconditionally categorize the indoor air quality of mass transportation enclosures as inadequate or unhealthy. For example, it has been established and documented that the transmission of serious contagious vaccine resistant strains of tuberculosis occurs while individuals are using public transport—i.e., within mass transportation enclosures. As another example, a study of the 2010 avian flu outbreak evaluated individuals who were hospitalized from the avian flu. The study showed that, in the aggregate, hospital workers were the number one group of hospitalized employment types by the flu followed very closely by transportation workers. These two groups made up a very large percentage of the total hospitalized population as a result of the flu. Such illness susceptibility data of transportation workers may be of little surprise when one recognizes that are exposed to large volumes of “conditioned” air or ventilation for long periods of time. Further, as one would recognize, non-worker users or patrons of transportation enclosures are exposed to the same large volumes of “conditioned” air or ventilation.

The exposure of large volumes of “conditioned” air or ventilation to users of transportation enclosures (both transportation workers and transportation patrons) poses a risk from not only naturally occurring or spreading illnesses, but from weaponized or purposefully occurring or spreading illnesses as well. For example, any microorganism or the like that causes disease or produces toxins can be used as a biological weapon.

The accessibility requirements of transportation enclosures, and the design and configuration of remote and often minimally protected storage facilities, provide for easy access to transportation enclosures. Tampering of HVAC systems or components with biological agents may easily allow for an act of terrorism or an otherwise sinister application of such biological agents. It is well known that public transportation is among the highest probable targets for bioterrorism activities. This concern has increased as smaller scale less structured terrorist groups and cells have been identified and successfully achieved their terrorism goals.

Microorganisms or other weaponized biological agents likely to be used in acts of bioterrorism are very effective in causing disease and illness. Such agents are usually produced or farmed from naturally occurring substances. The naturally occurring substances typically go through a variety of processes and procedures to transform them into an effective weaponized form. For example, typical bioterrorism agents are transformed through a powdering process to reduce the size range of the agent—usually making it smaller and more uniform (e.g., about 0.9 to 0.58 microns).

Currently used filters or like mechanisms in transportation enclosure HVAC systems are ineffective in filtering or otherwise preventing microorganisms, whether naturally occurring or weaponized, from users. Further, filtration methods that would effectively filter such microorganisms would require major redesign of transportation enclosures, and cause increased power demand for key HVAC components as well as a substantive increase in energy consumption. Unlike buildings and fixed facilities public transportation vehicles and systems are high volume vocationally designed and have limited flexibility in the alteration of the physical size, vehicle structure components and attributes, customer environment and integration into the normal transportation grid. In addition, the collection of live microorganisms on filters, in duct work for example, would create an extraordinary risk for employees, customers, first responders and eliminate the potential utilization of public transportation vehicles as shelter in place, evacuation or high volume people movers.

As result, a need exists for interior air treatment, control or quality systems for transportation enclosures that account for the variability in enclosure shape, available power, capacities, multiple functionalities and other vocational factors of transportation enclosures and that effectively decrease microorganism risk to users.

### BRIEF DESCRIPTION

In a first aspect, the disclosure provides an air treatment system for treating the air of a transportation enclosure that includes an air handling system with at least one plenum defining a flowpath of air through the air handling system and into the transportation enclosure via at least one input. The air treatment system includes a plurality of filters positioned, at least one ultraviolet (UV) light emitting device, and UV light reflective material within the flowpath. The plurality of filters include at least two filters increasing in ASHRAE Standard 52.2 MERV parameters in the direction of the flowpath of air. The filter positioned furthest downstream in the direction of flowpath of air is at least a parameter 8 MERV ASHRAE Standard 52.2 filter. The UV light reflective material is configured to increase the effective flux of the ultraviolet radiation of the at least one UV light emitting device within the flowpath. UV light emitted from the at least one UV light emitting device irradiates at least a portion of the filter positioned furthest downstream in the flowpath of air of the plurality of filters. The plurality of filters, at least one ultraviolet (UV) light emitting device, and UV light reflective material are operable to provide a dose of UV light of at least 20 J/m² to the input air flowing through the air treatment system at a first volumetric flow rate.

In some embodiments, the first volumetric flow rate may be less than about 700 cubic feet per minute. In some embodiments, the first volumetric flow rate may be less than about 550 cubic feet per minute. In some embodiments, the at least one UV light reflective material may be at least 50% UV light reflective.

In some embodiments, at least the filter positioned furthest downstream in the direction of flowpath of air, the at least one UV light emitting device, and the at least one UV light reflective material may be positioned within a treatment chamber, and the treatment chamber may be positioned between two portions of the at least one plenum such that the flowpath of air of the at least one plenum is directed to the treatment chamber and returned to the at least one plenum after flowing through the treatment chamber. In some embodiments, at least one UV light emitting device and the UV light reflective material may be configured to provide a dose of UV light of at least about 30 J/m² to the input air flowing through the air treatment system.

In some embodiments, the plurality of filters may include at least three filters positioned along the flowpath of air. In some such embodiments, the filter positioned furthest upstream in the flowpath of air may be positioned upstream of the at least one UV light emitting device. In some such embodiments, the filter positioned furthest upstream in the flowpath of air may be at least a parameter 4 MERV ASHRAE Standard 52.2 filter. In some such embodiments, the filter positioned furthest downstream in the flowpath of air may be at least a parameter 12 AS MERV ASHRAE Standard 52.2 filter, and a third filter positioned between the furthest upstream filter and furthest downstream filter in the flowpath of air may be at least a parameter 8 MERV ASHRAE Standard 52.2 filter.

In some embodiments, the filter positioned furthest downstream in the flowpath of air may be at least a parameter 12 MERV ASHRAE Standard 52.2 filter. In some embodiments, the filter positioned furthest downstream in the flowpath of air may be at least a parameter 13 MERV ASHRAE Standard 52.2 filter. In some embodiments, at least one of the plurality of filters, the at least one UV light emitting device, and at least one UV light reflective material may be positioned within a first duct, and the first duct may be positioned within the at least one plenum. In some such embodiments, the flowpath of air of the at least one plenum may be directed through the first duct.

In some embodiments, the filter positioned furthest downstream may extend at least partially about the at least one UV light emitting device. In some embodiments, UV light emitted from the at least one UV light emitting device may be ultraviolet germicidal irradiation (UVGI) or short-wavelength ultraviolet radiation (UV-C). In some embodiments, the total UV watt output of the at least one UV light emitting device may be at least about 30 UV watts. In some embodiments, at least one of the plurality of filters may include an anti-microbial coating. In some embodiments, the enclosure may be a railed or motor vehicle.

In another aspect, the discourse provides a transportation enclosure including an air treatment system as described above. In some embodiments, the transportation enclosure is a railed or motor vehicle.

In another aspect, the discourse provides a method for treating air of a transportation enclosure that includes an air handling system with at least one plenum defining a flowpath of air through the air handling system and into the transportation enclosure via at least one input. The method includes positioning a plurality of filters within the flowpath of air. At least two of the plurality of filters increase in ASHRAE Standard 52.2 MERV parameters in the direction of the flowpath of air. The filter positioned furthest downstream in the direction of flowpath of air is at least a parameter 8 MERV ASHRAE Standard 52.2 filter. The method further includes positioning at least one ultraviolet (UV) light emitting device within the flowpath of air and proximate at least the furthest downstream positioned filter of the plurality of filters in the direction of flowpath of air. The method further includes positioning UV light reflective material within the flowpath of air and about the at least one UV light emitting device to increase the effective flux of ultraviolet radiation emitted by the at least one UV light emitting device when energized within the flowpath of air. The method further includes energizing the at least one UV light emitting device such that the at least one UV light emitting device irradiates at least a portion of the furthest downstream positioned filter and provides a dose of UV light of at least 15 J/m² to the flowpath of air flowing at a first volumetric flow rate.

In some embodiments, the first volumetric flow rate may be less than about 700 cubic feet per minute. In some embodiments, the first volumetric flow rate may be less than about 550 cubic feet per minute. In some embodiments, the at least one UV light reflective material may be at least 50% UV light reflective. In some embodiments, the at least one UV light emitting device and the UV light reflective material may be configured to provide a dose of UV light of at least about 20 J/m² to the input air flowing through the air treatment system. In some embodiments, the at least one UV light emitting device and the UV light reflective material may be configured to provide a dose of UV light of at least about 30 J/m² to the flowpath of air.

In some embodiments, the plurality of filters may include at least three filters positioned along the flowpath of air. In some embodiments, the filter positioned furthest upstream in the flowpath of air may be positioned upstream of the at least one UV light emitting device. In some embodiments, the filter positioned furthest upstream in the flowpath of air may be at least a parameter 4 MERV ASHRAE Standard 52.2 filter. In some embodiments, the filter positioned furthest downstream in the flowpath of air may be at least a parameter 12 AS MERV ASHRAE Standard 52.2 filter, and a third filter positioned between the furthest upstream filter and furthest downstream filter in the flowpath of air may be at least a parameter 8 MERV ASHRAE Standard 52.2 filter. In some embodiments, the filter positioned furthest downstream in the flowpath of air may be at least a parameter 12 MERV ASHRAE Standard 52.2 filter. In some embodiments, the filter positioned furthest downstream in the flowpath of air may be at least a parameter 13 MERV ASHRAE Standard 52.2 filter.

In some embodiments, at least one of the plurality of filters, at least one UV light emitting device, and at least one UV light reflective material may be positioned within a first duct, and the first duct may be positioned within the at least one plenum such that the flowpath of air of the at least one plenum may be directed through the first duct. In some embodiments,

at least the filter positioned furthest downstream in the direction of flowpath of air, the at least one UV light emitting device, and the at least one UV light reflective material may be positioned within a treatment chamber. In some such embodiments the method may further include positioning the treatment chamber between two portions of the at least one plenum such that the flowpath of air of the at least one plenum extends through the treatment chamber.

In some embodiments, the filter positioned furthest downstream may extend at least partially about the at least one UV light emitting device. In some embodiments, at least one UV light emitted from the at least one UV light emitting device may be ultraviolet germicidal irradiation (UVGI) or short-wavelength ultraviolet radiation (UV-C). In some embodiments, the total UV watt output of the at least one UV light emitting device may be at least about 30 UV watts. In some embodiments, at least one of the plurality of filters may include an anti-microbial coating. In some embodiments, the enclosure may be a railed or motor vehicle.

In another aspect, the discourse provides an air treatment apparatus for treating the air of a transportation enclosure that includes an air handling system with at least one plenum defining a flowpath of air through the air handling system and into the transportation enclosure via at least one input. The air treatment apparatus includes a hollow treatment chamber, an input portion, an output portion, at least a first filter, at least one ultraviolet (UV) light emitting device, and UV light reflective material. The input portion is configured to couple to a first portion of the at least one plenum and direct the flow of air through the treatment chamber. The output portion is configured to couple to a second portion of the at least one plenum and direct the flow of air from the treatment chamber to the second portion of the at least one plenum. The first filter is positioned within the treatment chamber operable such that the flow of air through the treatment chamber flows through the first filter. The first filter is at least a parameter 12 MERV ASHRAE Standard 52.2 first filter. The at least one UV light emitting device is positioned within the treatment chamber. The UV light reflective material is positioned within the treatment chamber and is configured to increase the effective flux of the ultraviolet radiation of the at least one UV light emitting device within the treatment chamber. UV light emitted from the at least one UV light emitting device irradiates at least a portion of the first filter.

In some embodiments, the input portion, the first filter, and the at least one UV light emitting device may be provided on a sled member that is translatably coupled to treatment chamber. In some embodiments, the first filter, at least one UV light emitting device, and UV light reflective material may be operable to provide a dose of UV light of at least 20 J/m² to the flow of air through the treatment chamber.

In another aspect, the discourse provides an air treatment apparatus for treating the air of a transportation enclosure that includes an air handling system with at least one plenum defining a flowpath of air through the air handling system and into the transportation enclosure via at least one input. The air treatment apparatus includes a hollow treatment chamber, at least a first filter, at least one ultraviolet (UV) light emitting device, and UV light reflective material. The hollow treatment chamber includes an input portion configured to couple to a first portion of the at least one plenum and direct the flow of air through the treatment chamber, and an output portion configured to couple to a second portion of the at least one plenum and direct the flow of air from the treatment chamber to the second portion of the at least one plenum. The first filter is positioned within the treatment chamber and operable such that the flow of air through the treatment chamber flows through the first filter. The first filter is at least a parameter 8 MERV ASHRAE Standard 52.2 first filter. The at least one UV light emitting device is positioned within the treatment chamber. The UV light reflective material is positioned within the treatment chamber and configured to increase the effective flux of the ultraviolet radiation of the at least one UV light emitting device within the treatment chamber. UV light emitted from the at least one UV light emitting device irradiates at least a portion of the first filter. At least one of the input portion and the output portion includes a coupling including a hollow elastic receiver member that tapers outwardly from a first end to a second end as it extends in the direction of the flowpath of air. In some embodiments, the coupling further includes a hollow outer chamber, and wherein the second end of the receiver member is coupled to an inner surface of the outer chamber.

These and other objects, features and advantages of this disclosure will become apparent from the following detailed description of the various aspects of the disclosure taken in conjunction with the accompanying drawings.

### DRAWINGS

FIG. 1 illustrates a side cross-sectional view exemplary air treatment system for a transportation enclosure according to the present disclosure;

FIG. 2 illustrates a perspective view of a portion of the exemplary air treatment of FIG. 1;

FIG. 3 illustrates an exemplary air treatment system installed in an exemplary transportation enclosure according to the present disclosure;

FIG. 4 illustrates a side view of the exemplary air treatment system of FIG. 3;

FIG. 5 illustrates a perspective view of an exemplary air treatment system installed in another exemplary transportation enclosure according to the present disclosure;

FIG. 6A illustrates an enlarged elevational perspective view of the exemplary air treatment system of FIG. 5;

FIG. 6B illustrates a side view of the exemplary air treatment system of FIG. 5;

FIG. 7 is a perspective view of an exemplary air treatment apparatus for a transportation enclosure according to the present disclosure;

FIG. 8 is a perspective view of the air treatment apparatus of FIG. 7 illustrating internal components thereof;

FIG. 9 is a perspective exploded view of the air treatment apparatus of FIG. 7;

FIG. 10 is a top view of the air treatment apparatus of FIG. 7;

FIG. 11 is a side view of the air treatment apparatus of FIG. 7;

FIG. 12 is an end view of the air treatment apparatus of FIG. 7;

FIG. 13 is a perspective view of another exemplary air treatment apparatus for a transportation enclosure according to the present disclosure;

FIG. 14 is a perspective view of the air treatment apparatus of FIG. 13 illustrating internal components thereof;

FIG. 15 is a side view of the air treatment apparatus of FIG. 13;

FIG. 16 is an end view of the air treatment apparatus of FIG. 13; and

FIG. 17 is a top view of an exemplary transportation enclosure utilizing the air treatment apparatus of FIG. 7 and the air treatment apparatus of FIG. 13 in an air handling system.

### DETAILED DESCRIPTION

Each embodiment presented below facilitates the explanation of certain aspects of the disclosure, and should not be interpreted as limiting the scope of the disclosure. Moreover, approximating language, as used herein throughout the specification and claims, may be applied to modify any quantitative representation that could permissibly vary without resulting in a change in the basic function to which it is related. Accordingly, a value modified by a term or terms, such as “about,” is not limited to the precise value specified. In some instances, the approximating language may correspond to the precision of an instrument for measuring the value. When introducing elements of various embodiments, the articles “a,” “an,” “the,” and “said” are intended to mean that there are one or more of the elements. The terms “comprising,” “including,” and “having” are intended to be inclusive and mean that there may be additional elements other than the listed elements. As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances, the modified term may sometimes not be appropriate, capable, or suitable. Any examples of operating parameters are not exclusive of other parameters of the disclosed embodiments. Components, aspects, features, configurations, arrangements, uses and the like described, illustrated or otherwise disclosed herein with respect to any particular embodiment may similarly be applied to any other embodiment disclosed herein.

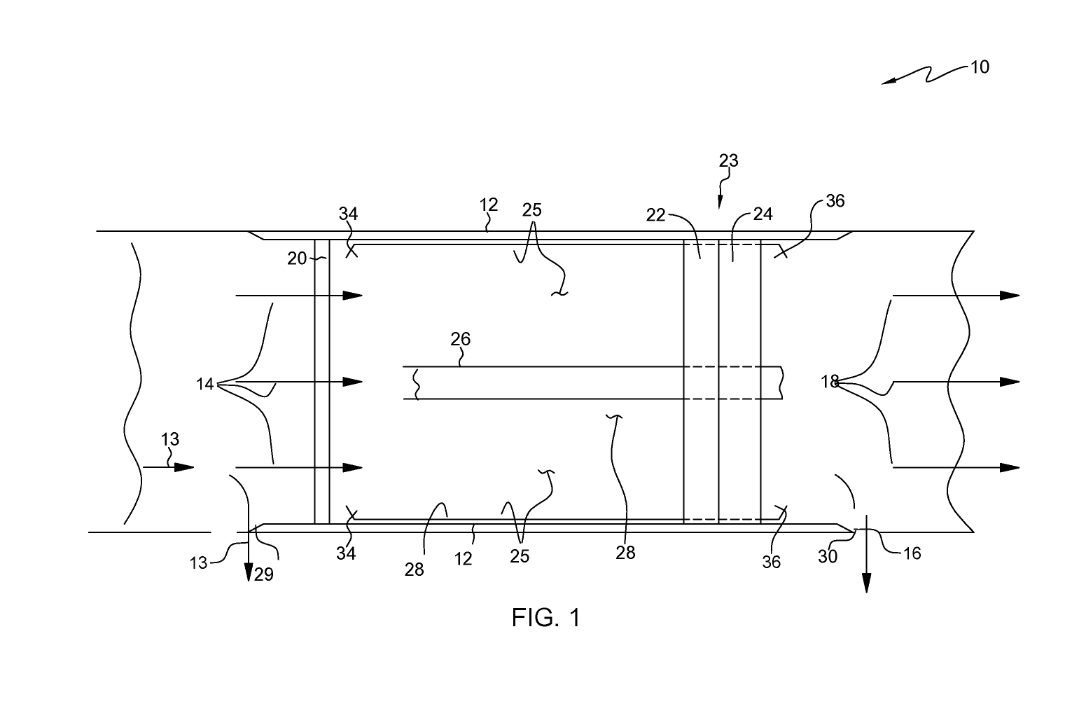

FIG. 1 illustrates an exemplary air treatment system generally referenced by the numeral 10 for a transportation enclosure according to the present disclosure. The exemplary air treatment system may be an interior air treatment, control or quality systems for transportation enclosures that accounts for the variability in enclosure shape, available power, capacities, multiple functionalities and other vocational factors of transportation enclosures, and that effectively decreases microorganism risk to users. In some embodiments, the air treatment system 10 deactivates, captures and neutralizes airborne aerosolized microorganisms and/or pathogens (such as potential biological weapons) in the air of air handling systems of a transportation enclosures to prevent such microorganisms and/or pathogens from circulating within the enclosures. In some embodiments, the air treatment system 10 is scalable and provides sufficient ultra violet (UV) light dose (or fluence) and filtration to various sized, shaped and otherwise engineered transportation enclosure air handling systems.

In some embodiments, the air treatment system 10 is configured to reduce the potential for, or frequency of, exposure of transportation enclosure users in all modes of transport, and to create barriers to the transmission of highly contagious diseases in those area where there are high densities of individuals who have compromised immune systems or who may be seriously affected by the exposure to any of the disease carrying microorganisms or pathogens that are commonly spread through airborne exposure. In some embodiments, the air treatment system 10 is configured to substantially eliminate, destroy, and/or capture those most probable agents of airborne terrorism in a relatively shortest duration of time—thereby limiting the exposure and potential casualty rate to transportation enclosure users. Such agents may be one or more agent identified as threats by the Department of Homeland Security, National Defense Administration, Center for Disease Control, EPA, and FEMA. Similarly, in some embodiments the air treatment system 10 is configured to remove allergens and other airborne particles and debris that are associated with asthma and other respiratory diseases from air within an air handling system of a transportation enclosure. In some embodiments, the air treatment system 10 is configured to allow the transportation enclosure to be used in contaminated areas for evacuation, sheltering in place and transport of remediation workers into a high risk area. In some embodiments, the air treatment system 10 is substantially integrated to system multiplex and subsystem connectivity of a transportation enclosure to provide for system prognostics, estimated efficiencies and maximum performance.

The transportation enclosure may be any transportation enclosure configured to transport a one or more, regardless of size, type or purpose. Users of the transportation enclosure may be patrons, customers or others utilizing the transportation enclosure for transportation between locations, or a worker or employee operating, maintaining or otherwise provided a function for the transportation enclosure in some manner. The transportation enclosure worker or employee may be any worker, such as a driver, engineer, maintenance worker, security personnel, hospitality personnel, etc. For example, the transportation enclosure may be a bus or other motor vehicle, railed vehicle, ferry, aircraft, tram, tractor, hovercraft, or any other enclosure operable to transport one or more user.

The transportation enclosure may be substantially enclosed or sealed such that an environment about the users is at least somewhat defined by the enclosure. For example, the transportation enclosure may be substantially enclosed but include windows, doors or other apertures through the enclosure that are open-able to the environment. Further, the enclosure may be any transportation enclosure with an air handling or ventilation system, such as a HVAC system. The air handling system of the enclosure may take any form, and may provide ventilation at ambient temperature, cooled air and/or heated air. The enclosure may include any of blowers, fans, cooling systems, heating systems, plenums, ducts, vents, intakes, temperature sensors, pressure sensors, other sensors, controls, and the like.

As shown in FIG. 1, the air treatment system 10 may be provided within a transportation enclosure including an air handling system with at least one plenum 12 defining a flowpath of air 14 through the air handling system and into the transportation enclosure via at least one input 16 at a first volumetric flow rate. In some embodiments, the transportation enclosure may include multiple plenums or duct branches, and the air treatment system 10 may be provided in multiple plenums or duct branches. For example, in some embodiments a transportation enclosure may include two main plenums or duct branches extending from a blower or other source air handling system component, and each of the two main plenums or duct branches may include the air treatment system 10. As another example, in some embodiments a transportation enclosures may include a main plenums or duct branch extending at opposing lateral side along the length of the transportation enclosure, and each side main plenums or duct branch of the transportation enclosure may include the air treatment system 10.

As shown in FIG. 1, in some embodiments the air treatment system 10 may be provided within the at least one plenum 12 of the transportation enclosure such that air 14 flowing through the at least one plenum 12 is forced or directed through a duct or other defined passageway 28 of the air treatment system 10, and then returned to the at least one plenum 12 of the transportation enclosure (or other air handling passageway of the transportation enclosure). In alternative embodiments, as described further below, the air treatment system 10 may be positioned exterior to the at least one plenum 12 of the transportation enclosure. In such embodiments, the air treatment system 10 may include a duct, chamber or the like that is in communication with the at least one plenum 12 and defines the passageway 28 such that air 14 flowing through the at least one plenum 12 is forced or directed through the duct, chamber or the like, and then returned to the at least one plenum 12 of the transportation enclosure. In this way, the air treatment system 10 may be spliced or interposed within the at least one plenum 12 (i.e., positioned between two portions of the at least one plenum 12) rather than being contained within the at least one plenum 12.

In some embodiments, the passageway 28 of the air treatment system 10 may be any air passageway effective in substantially directing and containing the input air 14, such as a substantially airtight passageway. In some embodiments, the passageway 28 may be a metal or plastic duct member. In some embodiments, the passageway 28 of the air treatment system 10 may be substantially contained within the at least one of a plenum 12 of the transportation enclosure. In other embodiments, the passageway 28 of the air treatment system 10 may be spliced or interposed between two portions the at least one of a plenum 12 of the transportation enclosure. As such, in some embodiments the passageway 28 of the air treatment system 10 may be substantially smaller in diameter, width, height and/or length of the corresponding plenum 12 of the transportation enclosure in which the passageway 28 is contained (i.e., smaller in length (direction of low) and at least one other direction. In some embodiments, the input air 14 may be funneled or otherwise constricted or directed into the relatively smaller passageway 28. In some other embodiments, the passageway 28 of the air treatment system 10 is positioned between or intermediate to the at least one of a plenum 12 of the transportation enclosure. For example, the passageway 28 of the air treatment system 10 may be positioned between or intermediate ends of a corresponding plenum 12 of the transportation enclosure in the direction of the flowpath of the input air 14. As such, in some embodiments the air treatment system 10 may not include a duct or passageway 28 itself, but rather make use of or incorporate a plenum, duct, or passageway of an already existing part or aspect of an air handling system of a transportation enclosure (e.g., the at least one plenum 12).

In some embodiments, the air treatment system 10 may be a retrofit system that is configured to be installed in an already manufactured or in-use transportation enclosure. For example, in some embodiments the air treatment system 10 may be a modification of an existing air handling system. As such, the present disclosure provides methods of retrofitting or modifying an air handling system of a transportation enclosure to treat the air provided to the enclosure by the air handling system. In some other embodiments, the air treatment system 10 is part of an as-manufactured transportation enclosure. For example, the air treatment system 10 may be a part or an aspect of the manufacturing process of the transportation enclosure itself As such, the present disclosure provides methods of manufacturing of a transportation enclosure with an air handling system that treats the air provided to the enclosure by the air handling system.

As also shown in FIG. 1, after the input air 14 is treated via the air treatment system 10, at least some of the treated air is input 16 into the enclosure by at least one input 30. On some embodiments, the at least one input 30 is a vent or other opening that allows treated input air 16 to flow into the enclosure. In some embodiments, other treated air 18 flows downstream past the at least one input 30 to other parts of the air handling system of the enclosure. In some embodiments, the at least one input 30 includes multiple inputs spaced downstream from the air treatment system 10 in the at least one plenum 12. In some such embodiments, the remaining treated air flows downstream and eventually enters the enclosure through the plurality of at least one inputs 30. As explained further below, in some embodiments the remaining treated air 18 flows downstream to further treatment aspects and to an area proximate and operator of the transportation enclosure.

In some embodiments, the air treatment system 10 is configured such that the input air 14 flowing into and through air treatment system 10 (and any microorganisms/pathogens contained therein), such as the input air 14 that receives a dose of UV light (as described further below), flows at a first volumetric flow rate of equal to or less than about 700 cubic feet per minute. It is noted, however, that microorganisms/pathogens and other particles may become trapped (at least temporarily) in at least of filter of the air treatment system 10 that is irradiated by at least one UV light emitting device 26, and thereby such particles may move more slowly through the air treatment system 10 (if at all) than the remainder of the input air 14 flowing through air treatment system 10. In some embodiments, the air treatment system 10 is configured such that the input air 14 flowing into and through air treatment system 10 (and any microorganisms/pathogens contained therein), such as the input air 14 that receives a dose of UV light (as described further below), flows at a first volumetric flow rate of equal to or less than about 630 cubic feet per minute. In some embodiments, the air treatment system 10 is configured such that the input air 14 flowing into and through air treatment system 10 (and any microorganisms/pathogens contained therein), such as the input air 14 that receives a dose of UV light (as described further below), flows at a first volumetric flow rate of equal to or less than about 550 cubic feet per minute. In some embodiments, the air treatment system 10 is configured such that the input air 14 flowing into and through air treatment system 10 (and any microorganisms/pathogens contained therein), such as the input air 14 that receives a dose of UV light (as described further below), flows at a first volumetric flow rate of equal to or less than about 480 cubic feet per minute. The volumetric flow rate of the flow input air 14 may be reduced before entering the treatment zone (i.e., UV dosage area) of the air treatment system 10 (as compared to a more upstream position) by restrictions within the air handling system and/or at least one plenum 12, at least one second input 29 that provides secondary input air 13 into the enclosure, one or more filters of the air treatment system 10, or any other mechanism effective in reducing the input air 14 flowing into and through air treatment system 10, such as to the above-described limits.

In some embodiments, the air treatment system 10 may be configured such that the air entering the enclosure downstream of the air treatment system 10 is at least about 2 and ½ cubic feet per minute for each user of the enclosure. In some embodiments, the air treatment system 10 may be configured to provide air into the enclosure downstream of the air treatment system 10 at a rate, volume, quantity, quality, temperature or the like that meets currently known and published transportation industry standards. In some embodiments, the air treatment system 10 may be configured using ASHRAE commercial HVAC system standards to limit flow restriction to input air 16 into the enclosure.

As described above, in some embodiments the air treatment system 10 may be configured to manage the flow rate of the input air 14, such as the input air 14 being irradiated by at least one UV light emitting device 26. For example, in some embodiments the air treatment system 10 may be configured limit the volumetric flow rate of the input air 14, such as the volumetric flow rate of the input air 14 being irradiated by at least one UV light emitting device 26, of less than or equal to about 700 cubic feet per minute. In some embodiments, the configuration of the filters of the air treatment system 10 (described below) may be a function of ducting 28 arrangement, proximity to plenum outlet (e.g., at least one input 30), velocity and volume of input air 14, as well as fan and fan/blower motor size and plenum 28 volume of the air handling system (such as to limit the flow rate of the input air 14 into and through the air treatment system 10 to an acceptable level). For example, in some embodiments the air treatment system 10 may be configured to normalize the flow rate and/or volume of the input air 14 before being treated by at least one UV light emitting device 26. In some embodiments, the air treatment system 10 (e.g., the filters of the air treatment system 10 (described below)) may result in a 5% or less increase in power demand of the motor of the air handling system of the enclosure. In some embodiments, the air treatment system 10 may result in a 3% or less increase in power demand of the motor of the air handling system of the enclosure.

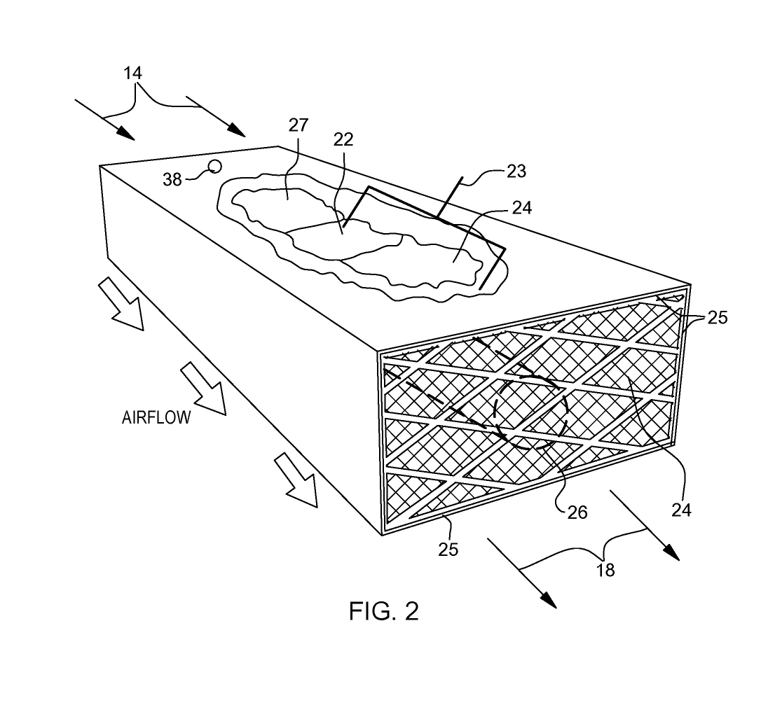

In some embodiments, the air treatment system 10 includes a first filter 20 configured such that the input air 14 is forced through the first filter 20. In some embodiments, substantially all of the input air 14 in forced or directed through the first filter 20. In some embodiments, the first filter 20 is positioned within the at least one plenum 12 of the air handling system of the enclosure (and, potentially, not in the passageway 28 of the air treatment system 10). In some embodiments, the first filter 20 is positioned within the passageway 28 of the air treatment system 10 (which may or may not be positioned within the at least one plenum 12, as described above). As shown in FIGS. 1 and 2, the first filter 20 may be configured such that the input air 14 is filtered by the first filter 20 before being treated by at least one UV emitting device 26. In some embodiments, the first filter 20 effectively reduces the flow rate and/or volume of the input air 14.

In some embodiments, the first filter 20 of the air treatment system 10 is configured to reduce relatively large particles or microorganism from the input air 14. For example, the air treatment system 10 may include additional filters, as discussed below, and the first filter 20 may allow the largest particles to pass through the filter as compared to the plurality of filters of the air treatment system 10. In some embodiments, the first filter 20 of the air treatment system 10 is at least a parameter 2 MERV ASHRAE Standard 52.2 filter. In some embodiments, the first filter 20 of the air treatment system 10 is at least a parameter 3 MERV ASHRAE Standard 52.2 filter. In some embodiments, the first filter 20 of the air treatment system 10 is at least a parameter 4 MERV ASHRAE Standard 52.2 filter.

TABLE 1
MERV Parameters—ASHRAE 52.2 Standard

| Standard 52.2 | Composite Average Particle Size Efficiency, % | | | | | |
|---|---|---|---|---|---|---|
| Minimum | in Size Range, μm | | | Average | | |
| Efficiency | Range | Range | Range | Arrestance, % | Minimum Final | |
| Reporting | Group 1 | Group 2 | Group 3 | by Standard | Resistance | |
| Value (MERV) | 0.30~1.0 | 1.0~3.0 | 3.0~10.0 | 52.1 Method | Pa | in. of water |
| 1 | n/a | n/a | E₃ < 20 | A_{avg} < 65 | 75 | 0.3 |
| 2 | n/a | n/a | E₃ < 20 | 65 ≦ A_{avg} < 70 | 75 | 0.3 |
| 3 | n/a | n/a | E₃ < 20 | 70 ≦ A_{avg} < 75 | 75 | 0.3 |
| 4 | n/a | n/a | E₃ < 20 | 75 ≦ A_{avg} | 75 | 0.3 |
| 5 | n/a | n/a | 20 ≦ E₃ < 35 | n/a | 150 | 0.6 |
| 6 | n/a | n/a | 35 ≦ E₃ < 50 | n/a | 150 | 0.6 |
| 7 | n/a | n/a | 50 ≦ E₃ < 20 | n/a | 150 | 0.6 |
| 8 | n/a | n/a | 70 ≦ E₃ | n/a | 150 | 0.6 |
| 9 | n/a | E₂ < 50 | 85 ≦ E₃ | n/a | 250 | 1.0 |
| 10 | n/a | 50 ≦ E₂ < 65 | 85 ≦ E₃ | n/a | 250 | 1.0 |
| 11 | n/a | 65 ≦ E₂ < 80 | 85 ≦ E₃ | n/a | 250 | 1.0 |
| 12 | n/a | 80 ≦ E₂ | 90 ≦ E₃ | n/a | 250 | 1.0 |
| 13 | E₁ < 75 | 90 ≦ E₂ | 90 ≦ E₃ | n/a | 350 | 1.4 |
| 14 | 75 ≦ E₁ < 85 | 90 ≦ E₂ | 90 ≦ E₃ | n/a | 350 | 1.4 |
| 15 | 85 ≦ E₁ < 95 | 90 ≦ E₂ | 90 ≦ E₃ | n/a | 350 | 1.4 |
| 16 | 95 ≦ E₁ | 95 ≦ E₂ | 95 ≦ E₃ | n/a | 350 | 1.4 |

It is noted that in some embodiments, the first filter 20 of the air treatment system 10 may be substantially spaced from other parts, components or aspects of the air treatment system 10. For example, the first filter 20 may be proximate the fan or blower of the air handling system of the transportation enclosure (e.g., within the at least one plenum 12), and other parts, components or aspects of the air treatment system 10 may be substantially downstream of the first filter 20. In some alternative embodiments (not shown), the first filter 20 of the air treatment system 10 may be provided at least partially upstream of at least one UV light emitting device 26. In some alternative embodiments (not shown), the air treatment system may not include the first filter 20.

As shown in FIG. 1, in some embodiments the air treatment system 10 may include UV light reflective material 25. For example, as shown in FIGS. 1 and 2, the air treatment system 10 may include UV light reflective material 25 about the flowpath of the input air 14 through the air treatment system 10. In some embodiments, the reflective material 25 may be adjacent the passageway 28 and/or plenum 12. In some embodiments, the reflective material 25 may be the inner surface of the passageway 28 and/or plenum 12. In some other embodiments, the reflective material 25 may be positioned within, or be the inner surface of, a duct, chamber or the like that is in communication with the at least one plenum 12 (e.g., spliced or interposed between two portions of the at least one plenum 12). In some embodiments, the reflective material 25 may be downstream of the first filter 20. In some embodiments, the reflective material 25 may be at least partially upstream of the additional filters of the air treatment system 10 (explained further below). In some embodiments, at least one UV light reflective material 25 may extend downstream and/or upstream past the upstream and downstream end, respectively of the light emitting device 26.

As shown in FIG. 1, in some at least one UV light reflective material 25 may form an upstream flange 34 or like shape and/or a downstream flange 36 at the ends of the reflective material 25 along the direction of the flow of the air through the air treatment system 10. In some embodiments, the upstream flange 34 and/or a downstream flange 36 of the reflective material 25 may be positioned upstream or downstream, respectively, of at least one UV light emitting device 26, as shown in FIG. 1. In some embodiments, the upstream flange 34 and/or a downstream flange 36 of at least one UV light reflective material 25 of the air treatment system 10 may be directed to reflect at least a portion of UV light emitted from at least one UV light emitting device 26 inwards toward a medial portion of the reflective material 25 such that at least a portion of at least one UV light is prevented from exiting the ends of the reflective material 25 along the direction of the flow of the air through the air treatment system 10.

In some embodiments, at least one UV light reflective material 25 within the at least one plenum 12 and/or passageway 28 may be configured to increase the intensity or flux density of the ultraviolet radiation of at least one UV light emitting device 26. For example, in some embodiments at least one UV light reflective material 25 may provide additional UV light intensity from reflections and inter-reflections of at least one UV light emitted from at least one UV light emitting device 26. The UV light reflective material 25 may be configured to reflect UV light emitted from at least one UV light emitting device 26 and thereby irradiate additional, or an increased density of, UV light onto the input air 14 flowing through the air treatment system 10. For example, at least one UV light reflective material 25 may substantially completely surround or extend about at least one UV light emitting device 26 as it extends along the flow path, and potentially extend further upstream and/or downstream past the upstream and downstream ends, respectively, of at least one UV light emitting device 26. In some embodiments, at least one UV light reflective material 25 may extend about at least one UV light emitting device 26 such that opposing sides or surfaces of at least one UV light reflective material 25 are positioned equidistant from at least one UV light emitting device 26. Stated differently, at least one UV light emitting device 26 may be substantially centered within at least one UV light reflective material 25 (e.g., opposing sides of at least one UV light emitting device 26, such as a bulb, are equidistant to at least one UV light reflective material 25).

In some embodiments, at least one UV light reflective material 25 is a substantially UV impenetrable member or material. In some embodiments, at least one UV light reflective material 25 is a metal material. In some embodiments, at least one UV light reflective material 25 is a galvanized metallic duct. In some embodiments, at least one UV light reflective material 25 is an aluminum foil. In some embodiments, at least one UV light reflective material 25 is an aluminum treated surface. In some embodiments, at least one UV light reflective material 25 is an etched aluminum surface.

In some embodiments, at least one UV light reflective material 25 may reflect UV light. In some such embodiments, at least one UV light reflective material 25 may substantially reflect UV light in the b-band (UV-B) and c-band (UV-C)—about 315 to about 100 nanometer wavelength light. In some embodiments, at least one UV light reflective material 25 may reflect at least about 50% UV-C and/or UV-B light. In some embodiments, at least one UV light reflective material 25 may reflect at least about 50% short-wavelength ultraviolet radiation (UV-C light). In some embodiments, at least one UV light reflective material 25 may reflect at least about 57% UV-C light. In some embodiments, at least one UV light reflective material 25 may reflect at least about 73% UV-C light. In some embodiments, at least one UV light reflective material 25 may reflect at least about 74% UV-C light. In some embodiments, at least one UV light reflective material 25 may reflect at least about 78% UV-C light. In some embodiments, at least one UV light reflective material 25 may reflect at least about 88% UV-C light. In some embodiments, at least one UV light reflective material 25 may reflect at least about 90% UV-C light.

As explained above and shown in FIGS. 1 and 2, the air treatment system 10 may include at last one UV light emitting device 26. In some embodiments, at least one UV light emitting device 26 may be positioned within the at least one plenum 12 and/or passageway 28 of the air treatment system 10. In some other embodiments, the at least one UV light emitting device 26 may be positioned within, or be the inner surface of, a duct, chamber or the like that is in communication with the at least one plenum 12 (e.g., spliced or interposed between two portions of the at least one plenum 12). As also explained above, at least one UV light emitting device 26 may be substantially surrounded (e.g., along a plane normal to the flow path of air through the air treatment system 10) by UV light reflective material 25. In some embodiments, at least one UV light emitting device 26 may be suspended within the at least one plenum 12, passageway 28, and/or UV light reflective material 25 such that the input air 14 is forced about at least one UV light emitting device 26 and between at least one UV light emitting device 26 and at least one UV light reflective material 25.

In some embodiments, at least one UV light emitting device 26 may be extended along the direction of the flowpath of air through the through the air treatment system 10. In some embodiments, at least one UV light emitting device 26 may be extended along a direction angled from the direction of the flowpath of air through the through the air treatment system 10, such as substantially perpendicular. In some embodiments, the total linear length of the at least one UV light emitting device 26 of the air treatment system 10 may be at least about 12 inches. In some embodiments, the total linear length of the at least one UV light emitting device 26 of the air treatment system 10 may be at least about 24 inches. In some embodiments, the total linear length of the at least one UV light emitting device 26 of the air treatment system 10 may be at least about 36 inches. In some embodiments, the total linear length of the at least one UV light emitting device 26 of the air treatment system 10 may be at least about 42 inches. In some embodiments, the total linear length of the at least one UV light emitting device 26 of the air treatment system 10 may be at least about 48 inches. In some embodiments, the total linear length of the at least one UV light emitting device 26 of the air treatment system 10 may be at least about 72 inches. In some embodiments, the total linear length of the at least one UV light emitting device 26 of the air treatment system 10 may be at least about 96 inches. In some embodiments, as described further below, the total linear length of the at least one UV light emitting device 26 specifically for the operator of the enclosure may be at least 12 inches in length. It is noted that in some embodiments the air treatment system 10 may include a plurality of UV light emitting devices 26, and such UV light emitting devices 26 may be stacked or adjacent across the direction of flow (i.e., not end to end along the direction of flow). In some embodiments, the at least one UV light emitting device 26 of the air treatment system 10 may be a plurality of non-linear extended bulbs or devises. For example, the at least one UV light emitting device 26 of the air treatment system 10 may be plurality of relatively small UV-LED devices.

In some embodiments, at least one UV light emitting device 26 may be extendable along the direction of the flowpath of air through the through the air treatment system 10. In some embodiments, at least one UV light emitting device 26 may include a plurality of UV light emitting devices spaced or positioned along the direction of the flowpath of air through the through the air treatment system 10. In some embodiments, at least one UV light emitting device 26 may include a plurality of UV light emitting devices spaced or positioned along a plane positioned normal to the flowpath of air through the through the air treatment system 10. In some embodiments, at least one UV light emitting device 26 may be positioned at least partially downstream in the flowpath of air from at least one of the plurality of filters of the air treatment system 10.

The at least one UV light emitting device 26 may be any device or combination of devices effective in emitting UV light. For example, at least one UV light emitting device 26 may be any UV emitting bulb or like device. In some embodiments, the at least one UV light emitting device 26 may be at least one UV-LED device. In some embodiments, the at least one UV light emitting device 26 may emit ultraviolet germicidal irradiation (UVGI)—UV light at sufficiently short wavelengths to kill or inactivate microorganisms. In some such embodiments, at least one UVGI light may be short-wavelength ultraviolet radiation or light (UV-C). In some embodiments, the at least one UV light emitting device 26 may emit light with wavelengths at least about 100 nm. In some embodiments, the at least one UV light emitting device 26 may emit light with a wavelength of about 254 nm. In some embodiments, the at least one UV light emitting device 26 may emit light with wavelengths within the range of about 224 nm to about 284 nm, or within the range of about 244 nm to about 264 nm. In some embodiments, the at least one UV light emitting device 26 may emit light with wavelengths at within the range of about 249 nm to about 259 nm.

In some embodiments, the total UV watt output (radiant power or flux) by the at least one UV light emitting device 26 of each air treatment system 10 may be at least 10 UV Watts. In some embodiments, the total UV watt output (radiant power or flux) by the at least one UV light emitting device 26 of each air treatment system 15 may be at least 15 UV Watts. In some embodiments, the total UV watt output (radiant power or flux) by the at least one UV light emitting device 26 of each air treatment system 18 may be at least 18 UV Watts. In some embodiments, the total UV watt output (radiant power or flux) by the at least one UV light emitting device 26 of each air treatment system 30 may be at least 30 UV Watts.

In some embodiments, the air treatment system 10 may include a shroud or shield to the at least one UV light emitting device 26, such as a plastic or metal shield, configured to prevent shattering and distribution of breakage shards of at least one UV light emitting device 26 in case of catastrophic failure or breakage. In some embodiments, the air treatment system 10 may include external UV resistant insulating material surrounding, at least partially, the circumference of the housing of at least one UV light emitting device 26 to limit the cooling effect on at least one UV light emitting device 26 from the exterior—which may lower at least one UV wattage and life of at least one UV light emitting device 26.

As shown in FIGS. 1 and 2, the air treatment system 10 may include at least one downstream filter 23 positioned at least partially downstream of the at least one UV light emitting device 26 in the direction of flow of the input air 14 through the air treatment system 10. For example, the air treatment system 10 may include at least one downstream filter 23 positioned downstream of the at least one UV light emitting device 26 in the direction of flow of the input air 14 through the air treatment system 10. As another example, the air treatment system 10 may include at least one downstream filter 23 formed or provided about a distal or downstream end or portion of the at least one UV light emitting device 26 in the direction of flow of the input air 14 through the air treatment system 10. In some embodiments, the air treatment system 10 may include at least one downstream filter 23 positioned at least partially downstream of the at least one UV light emitting device 26 and positioned between within the reflective material 25. In some embodiments, the air treatment system 10 may include at least one downstream filter 23 positioned within the at least one plenum 12 and/or the passageway 28. In some other embodiments, the at least one downstream filter 23 may be positioned within a duct, chamber or the like that is in communication with the at least one plenum 12 (e.g., spliced or interposed between two portions of the at least one plenum 12). In some embodiments, as shown in FIGS. 1 and 2, the air treatment system 10 may be configured that the input air passing through the air treatment system 10 is forced or otherwise directed through the at least one downstream filter 23. As such, the input air 14 may be directed through the first filter 20 (potentially), about the at least one UV light emitting device 26 and thereby between at least one UV light emitting device 26 and reflective material 25, and through the at least one downstream filter 23 before being output 18 by the air treatment system 10 and (and input 16 into the enclosure).

In some embodiments, the air treatment system 10 is configured such that UV light emitted from the at least one UV light emitting device 26 irradiates, or acts upon, the at least one downstream filter 23. For example, the air treatment system 10 may be configured such that UV-C light emitted from the at least one UV light emitting device 26 directly irradiates on the at least one downstream filter 23, and/or UV-C light emitted from the at least one UV light emitting device 26 indirectly irradiates on the at least one downstream filter 23 (e.g., at least one UV reflective material 25 reflects UV-C light emitted from the at least one UV light emitting device 26 onto the at least one downstream filter 23).

In some embodiments, the at least one downstream filter 23 may include a second downstream positioned filter 24 of the air treatment system 10 at least adjacent the at least one UV light emitting device 26. In some embodiments, the second filter 24 of the air treatment system 10 may be more fine (i.e., configured to trap finer particles or microorganisms) than the first filter 20 positioned upstream of the second filter 24 (if provided). In some embodiments, the second filter 24 may be at least a parameter 8 MERV ASHRAE Standard 52.2 filter. In some embodiments, the second filter 24 may be at least a parameter 10 MERV ASHRAE Standard 52.2 filter. In some embodiments, the second filter 24 may be at least a parameter 12 MERV ASHRAE Standard 52.2 filter. In some embodiments, the second filter 24 may be at least a parameter 13 MERV ASHRAE Standard 52.2 filter. It is noted again that UV light emitted from the at least one light emitting device 26 may emit irradiate (directly and/or indirectly) UV light on, and at least partially through, the second filter 24. In this way, microorganism, pathogens or the like trapped by the filter 24 are exposed to at least one UV light for a significant amount of time (i.e., receive a very high dose of UVGI, such as at least about 20 J/m²).

As shown in FIGS. 1 and 2, in some embodiments the at least one downstream filter 23 may include a third filter 22 of the air treatment system 10 positioned upstream of the second filter 24. In some embodiments, the second filter 24 and the third filter 24 may form a filter pack. In some embodiments, the third filter 22 may be positioned between the first filter 20 (if provided) and the second filter 24 in the direction of the flowpath of air through the air treatment system 10. In some embodiments, the third filter 22 may be at least about a parameter 8 MERV ASHRAE Standard 52.2 filter. In some embodiments, the third filter 22 may be at least about a parameter 9 MERV ASHRAE Standard 52.2 filter. In some embodiments, the second filter 24 may be at least about a parameter 10 MERV ASHRAE Standard 52.2 filter. It is noted again that UV light emitted from the at least one light emitting device 26 may emit irradiate (directly and/or indirectly) UV light on, and at least partially through (and thereby potentially to the second filter 24), the third filter 22. In this way, microorganism, pathogens or the like trapped by the third filter 22 are exposed to at least one UV light for a significant amount of time (i.e., receive a very high dose of UVGI, such as at least about 20 J/m²).

In some embodiments, the at least one downstream filter 23 may include a fourth filter 24 of the air treatment system 10 adjacent the third filter 22. In some such embodiments, the fourth filter 24 may be substantially similar to the first upstream filter 20. In some such embodiments, the fourth filter 24 is provided instead of the first upstream filter 20.

As recognized by on skill in the art, the present disclosure provides air treatment systems that deactivate, capture and neutralize airborne aerosolized microorganisms and/or pathogens (such as potential biological weapons) in the air handling system of a transportation enclosure to prevent such microorganisms and/or pathogens from circulating within the enclosure. For example, in some embodiments the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) of at least 15 J/m². In some embodiments, the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) of at least 20 J/m². In some embodiments, the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) of at least 30 J/m². In some embodiments, the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) of at least 40 J/m². In some embodiments, the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) of at least 100 J/m².

In some embodiments, the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) sufficient to kill or inactivate at least 90% of influenza, small pox, tuberculosis, and other microorganism/pathogens contained within the input air 14 (i.e., within one pass or cycle of air through the air treatment system 10) (e.g., a dose of at least 20 J/m²). In some embodiments, the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) sufficient to kill or inactivate at least 99% of influenza, small pox, tuberculosis, and other microorganism/pathogens contained within the input air 14 (i.e., within one pass or cycle of air through the air treatment system 10) (e.g., a dose of at least 40 J/m²). In some embodiments, the air treatment system 10 may be configured such that input air 14 flowing into and through the air treatment system 10 and eventually output by the system 10 and input into the enclosure (i.e., the output air 18 and input air 16) is treated by the air treatment system 10 with a dose of UV light (e.g., UVGI or UV-C) sufficient to kill or inactivate at least 49% of anthrax and other microorganism/pathogens contained within the input air 14 (i.e., within one pass or cycle of air through the air treatment system 10, and at least 98% within two passes or cycles of air through the air treatment system 10) (e.g., a dose of at least 100 J/m²).

It is noted that the air treatment systems of the present disclosure may be capable of providing the dosages described above due to the reduced flow rate of the input or treated air, the pre-filtering with the first filter, the size and power of at least one UV light emitting device, the reflectivity of the reflective material, the positing and filtering efficiency of the at least one downstream filter, and/or combinations thereof. Stated differently, the air treatment systems of the present disclosure may utilize filtration, UVGI, and low air flow rates to substantially mitigate damage from microbes, such as microbes of less than 0.5 microns in diameter. The combined application of UVGI, filtration and lower flow rate provide a relatively high probability of success for the inactivation of microbes. For example, while anthrax spores are easily filtered, they are extremely resistant to UVGI except at extremely high doses. As anthrax spores may be trapped and held in place by the at least one downstream filter 23, anthrax spores within the at least one downstream filter 23 would receive an extremely high dose of UVGI. As another example, small pox is highly penetrable to virtually all filtered media, but is very susceptible to UVGI. The low flow rate and relatively powerful UVGI may sufficiently kill or inactivate small pox. As yet another example, TB bacilli are mid-sized bacteria that are inactivated relatively easily by filtration or UVGI. Similarly, various influenza types or viruses and botulism toxin are extremely susceptible to UVGI.

In some embodiments, the air treatment system 10 may be configured such that the at least one UV light emitting device 26 remains active and emitting UV light that irradiates the at least one downstream filter 23 for a set period of time (e.g., 5 minutes) after the transportation enclosure is powered down or off. In some embodiments, the air treatment system 10 may be configured to perform a self-health check. In some embodiments, the air treatment system 10 may be configured to prevent access to the at least one UV light emitting device 26 and or at least one of the first filter 20 and a least one downstream filter 23 while at least one UV light emitting device 26 is energized.

In some embodiments, the air treatment system 10 may be configured such that at least one of the filters of the air treatment system 10 (e.g., at least one of the first filter 20 and the at least one downstream filter 23) includes an anti-microbial coating. In some such embodiments, the anti-microbial coating may be configured to attract microbes, pathogens and the like, and/or kill or deactivate such substances. In some embodiments, the at least one of the filters of the air treatment system 10 includes a titanium dioxide coating.

In some embodiments, the air treatment system 10 may include a separate UV plenum or duct, filter, and/or UV light emitting device) downstream of the at least one downstream filter 23 that provides treated air to the enclosure operator. In some embodiments, at least one UV plenum or duct and/or filter may reduce the flow rate of air to the operator. In some such embodiments the separate UV plenum or duct and filter may be void of a bypass in the duct or include openings. In some such embodiments, the operator specific duct, filter, and/or UV light emitting device may ensure the absolute minimum in exposure of the operator to any airborne illnesses and diseases. The position of this fixture will prevent re-breathing of contaminated air by the individual responsible for the movement of the enclosure, for example. This additional precaution will improve the likelihood of safe evacuation of the enclosure and users should that be necessary and provide the capability to “shelter in place.” In addition to reducing the flow rate of conditioned air to the operator's compartment and further treating such air, the air treatment system 10 may include a diverter duct system which segregates airflow to the operator so as to better disperse air in the operators compartment.

In some embodiments, the air treatment system 10 may include a diverter pathway (not shown) that is capable of directing the input air around or downstream at least some of the plurality of filters, reflective material and/or UV light emitting devices(s), but eventually into the enclosure via at least one input. Such bypass feature may be trigger by one or more sensor 37 in the flowpath of the air through or to the air treatment system 10 and/or monitoring the performance of one or more aspect of the air treatment system 10 and/or the enclosure, as shown in FIG. 2. For example, in some embodiments, the air treatment system 10 may include high back pressure and or temperature sensor that, when triggered, bypasses least some of the plurality of filters, reflective material and/or UV light emitting devices(s). In some such embodiments, of a filter of the plurality of filters may cause such high back pressure that triggers a bypass of at least one of the air treating mechanisms of the air treatment system 10.

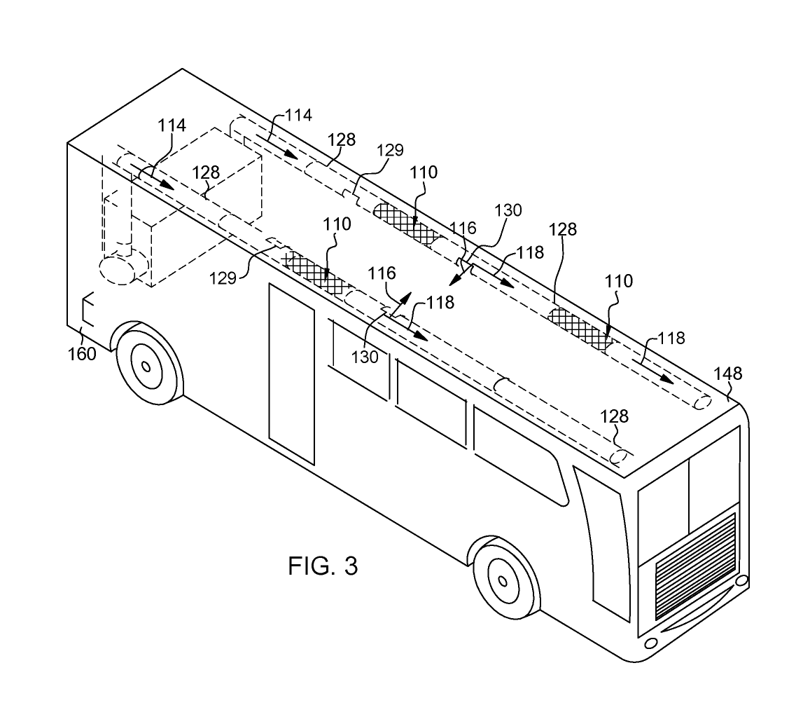

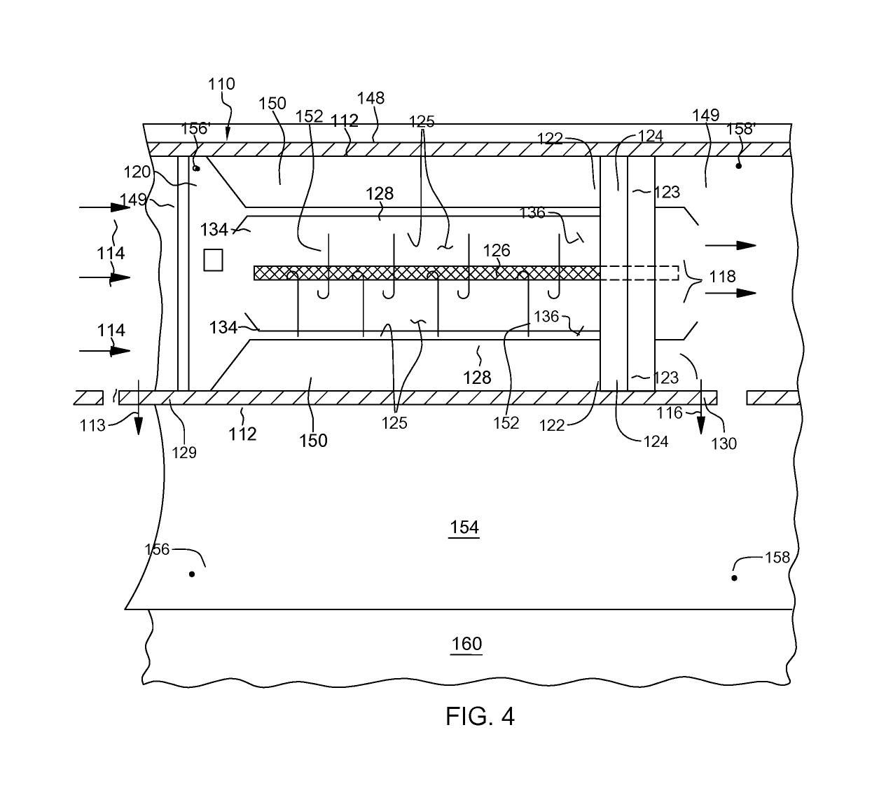

In FIGS. 3 and 4, another exemplary air treatment system of the present disclosure is indicated generally by the reference numeral 110. The air treatment system 110 is substantially similar to the air treatment system 10 described above with reference to FIGS. 1 and 2, and therefore like reference numerals preceded by the numeral “1” are used to indicate like elements, functions, aspects or the like. As shown in FIGS. 3 and 4, the air treatment system 110 differs from air treatment system 10 in the installation of the air treatment system 110 in a bus or similar transportation enclosure 160.

As shown in FIGS. 3 and 4, the air treatment system 110 may be installed within at least one plenum 112 of the air handing system of the bus 160. In the exemplary embodiment shown in FIGS. 3 and 4, the at least one plenum 112 is provided adjacent the roof or ceiling structure 148. In some embodiments, the gap or space between the plenum 112 and the roof or ceiling structure 148 may be substantially sealed or blocked off at either end in the direction of flow with one or more end cap. In the exemplary embodiment shown in FIGS. 3 and 4, the at least one plenum 112 is provided adjacent the roof or ceiling structure 148. For example, the at least one plenum 112 may be provided as a bulk head or the like adjacent or proximate the roof or ceiling structure 148 of the bus 160. In some embodiments, the at least one plenum 112 may be provided proximate the junction of the roof or ceiling structure 148 and a side wall of the bus 160. In some embodiments, the lateral sides of the bus 160 each include a plenum 112 extending the length of the bus 160 in the flow direction of the input and output air 114, 118, and each plenum 112 at each lateral side of the bus 160 may include the air treatment system 110. Further, as described above, the driver's lateral side of the bus 160 may include a driver's plenum, filter and/or UV light emitting device to provide a redundant air treatment system for the air provided to the operator.

As shown in FIG. 4, the passageway 128 of the air treatment system 110 may be supported or suspended inwardly from the plenum 112 and UV reflective material 125 by support brackets or members 150. In some embodiments, the gap or space between the plenum 112 and the passageway 128 may be substantially sealed or blocked off at either end in the direction of flow with one or more end cap. In some embodiments, the support brackets or members 150 may be any mechanism effective in supporting and/or defining the treatment passageway 128 though the plenum 112 and reflective material 125. As also shown in FIG. 4, the at least UV light emitting device 126 may be suspended or supported within a central portion of the treatment passageway 128 and reflective material 125 by exemplary hanger members 152. In some embodiments, the exemplary hanger members 152 may extend from a plurality of edges or sides of the treatment passageway 128 and reflective material 125 and support a side of the at least UV light emitting device 126. In some embodiments, the hanger members 152 may be resilient, deformable or spring-like. In this way, the at least UV light emitting device 126 may be resiliently suspended within the treatment passageway 128 and UV reflective material 125 such that the input air 14 may flow about the at least UV light emitting device 126 and between the at least UV light emitting device 126 and the reflective material 125. Further, as the at least UV light emitting device 126 may be resiliently supported by the hanger members 152, shock, vibration and other forces may be dampened or otherwise prevented from acting on and/or damaging the at least UV light emitting device 126.

It is noted that at least one UV light emitting device 126 and/or UV reflective material 125 may or may not extend at least partially into, or past, the at least one downstream filter 123. Such potential embodiments are shown with dashed lines in FIG. 4.

As also shown in FIG. 4, the compartment or space in the bus in which the plenum 112 and air treatment system 110 is contained may be closed off or sealed between the roof structure 148 and side wall 149 of the bus 160, for example, and a movable cover member 160. The cover member 160 may provide access to the plenum 112 and air treatment system 110 from within the bus 160, as shown in FIG. 4. In some embodiments, the cover member 160 may be hinged to provide a door-like cover member 160. As shown in FIG. 4, the cover member 160 may include first and second latches or like mechanisms 156, 158. The first and second latching mechanisms 156, 158 of the cover member 160 may be configured to lockably mate with corresponding first and second latching mechanisms 156′, 158′ provided on the roof structure 148 or other portion of the bus 160. In this way, the first and second latching mechanisms 156, 158 of the cover member 160 and the first and second latching mechanisms 156′, 158′ of the bus 160 may secure the cover member 160 over the air treatment system 110 and provide only limited access to the air treatment system 110. For example, the first and second latching mechanisms 156, 158 of the cover member 160 and the first and second latching mechanisms 156′, 158′ of the bus 160 may automatically lock during operation of the bus 160, and/or for a fixed timer period after the bus 160 stops operating. As another example, the first and second latching mechanisms 156, 158 of the cover member 160 and the first and second latching mechanisms 156′, 158′ of the bus 160 may require a key or other access limiting device to prevent non-authorized user's from accessing the air treatment system 110.

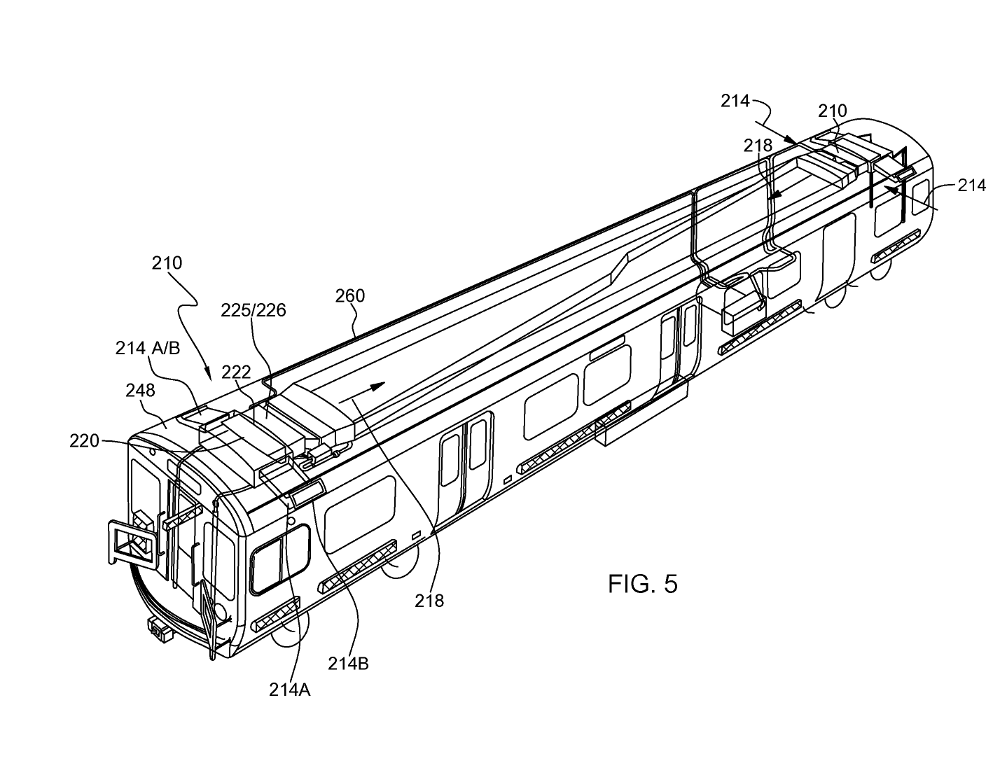

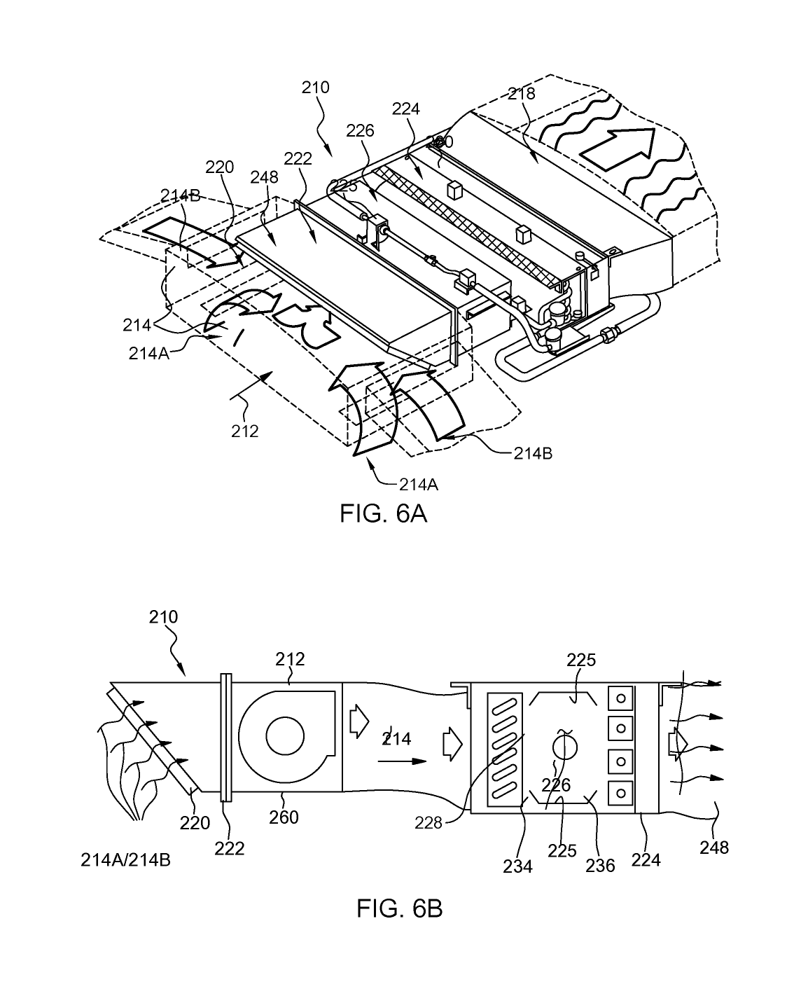

In FIGS. 5-6B, another exemplary air treatment system of the present disclosure is indicated generally by the reference numeral 210. The air treatment system 210 is substantially similar to the air treatment systems 10 and 110 described above with reference to FIGS. 1-4, and therefore like reference numerals preceded by the numeral “2” are used to indicate like elements, functions, aspects or the like. As shown in FIGS. 5-6B, the air treatment system 210 differs from air treatment systems 10 and 110 in the installation of the air treatment system 210 in a rail car or similar transportation enclosure 260.

As shown in FIGS. 5-6B, the air treatment system 210 may be installed within at least one plenum 212 of the air handing system of a rail car 260. In the exemplary embodiment shown in FIGS. 5-6B, the at least one plenum 212 is positioned proximate the roof 249 of the rail car 260 at the rear of the rail car 260. As shown in FIGS. 5-6B, the input air 214 may comprise both recycled input air 214A that is pulled or taken from within the rail car 260 and fresh input air 214 that is pulled form exterior to the rail car 260 (e.g., from the environment). As also shown in FIGS. 5-6B, the air treatment system 210 may include the first filter 220 and third filter 222 upstream of the at least one UV emitting device 226 and UV reflective material 225. In some embodiments, the air treatment system 210 may include the first filter 220 and third filter 222 upstream of at least one fan or blower configured to move air throughout the at least one plenum 212. As also shown in FIGS. 5-6B, the air treatment system 210 may include the second filter 224 downstream, at least partially, of the at least one UV emitting device 226 and UV reflective material 225 (and first filter 220 and third filter 222). In some such embodiments, at least one UV light emitted from the at least one UV emitting device 226 and reflected by at least one UV reflective material 225 may substantially irradiate on the second filter 224 (and the air flow within the passageway 228).

In some embodiments, as shown in FIGS. 5-6B, the at least one UV emitting device 226 of the air treatment system 210 may me substantially laterally elongated across the width of the at least one plenum 212 and direction of the flowpath of air through the air treatment system 210, rather than, or in addition to, being longitudinally elongated along the direction of the flowpath of air through the air treatment system 210. In some such embodiments, the at least one UV emitting device 226 of the air treatment system 210 may include a plurality of substantially laterally elongated UV emitting devices.

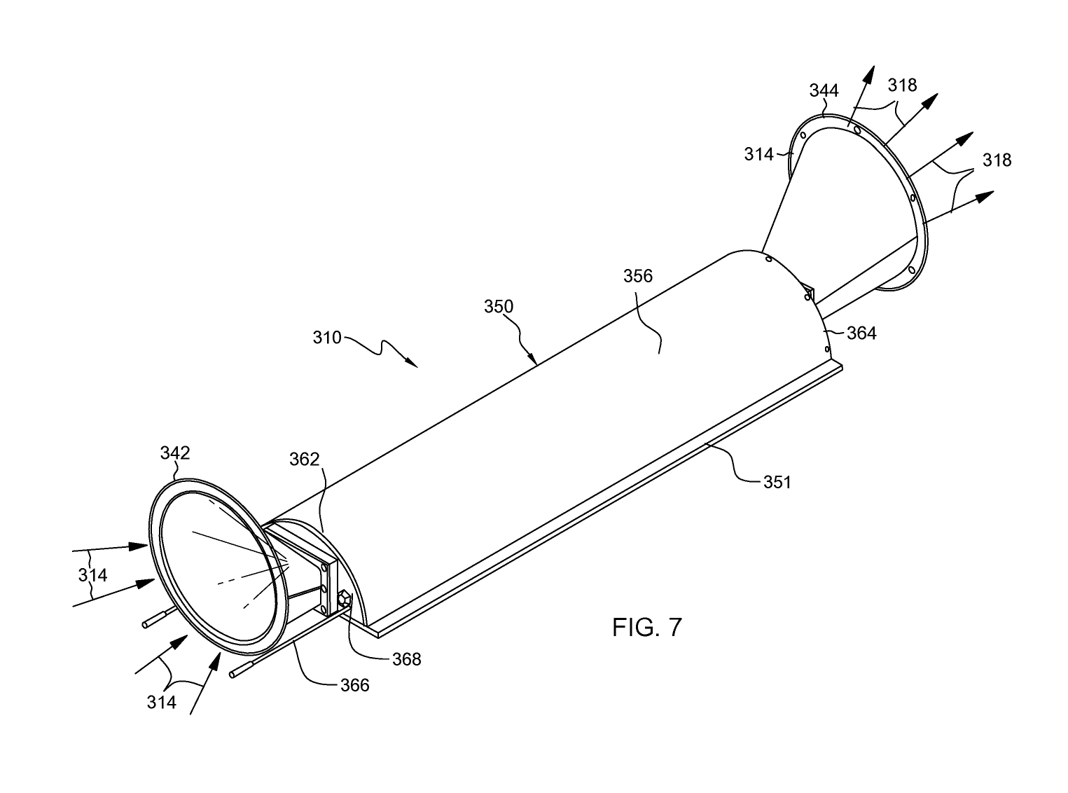

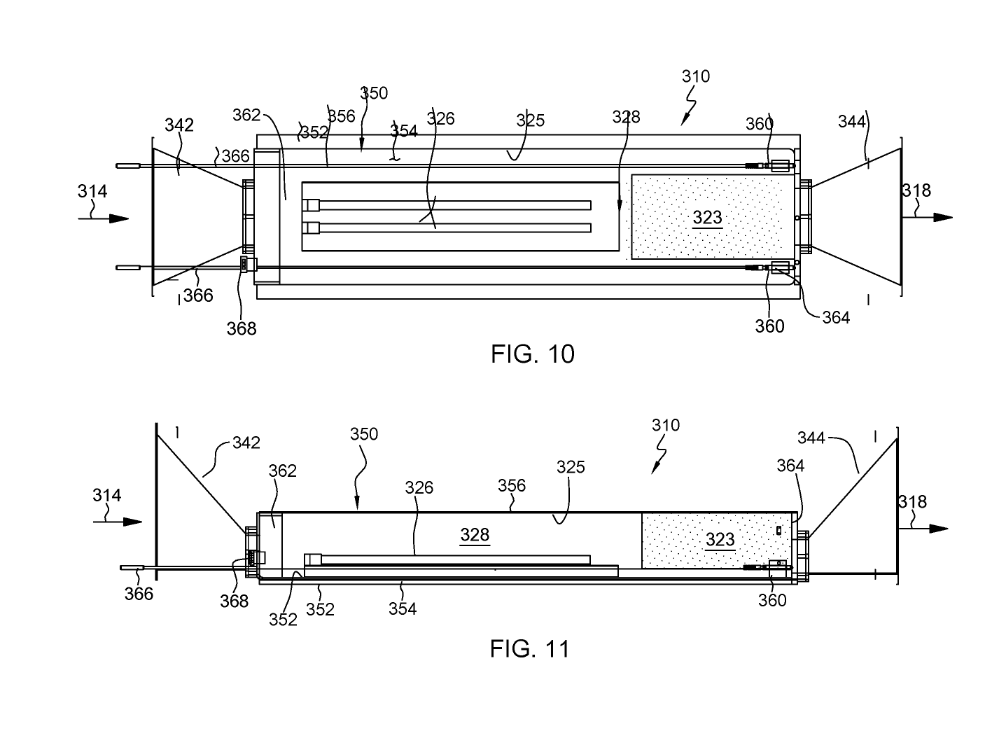

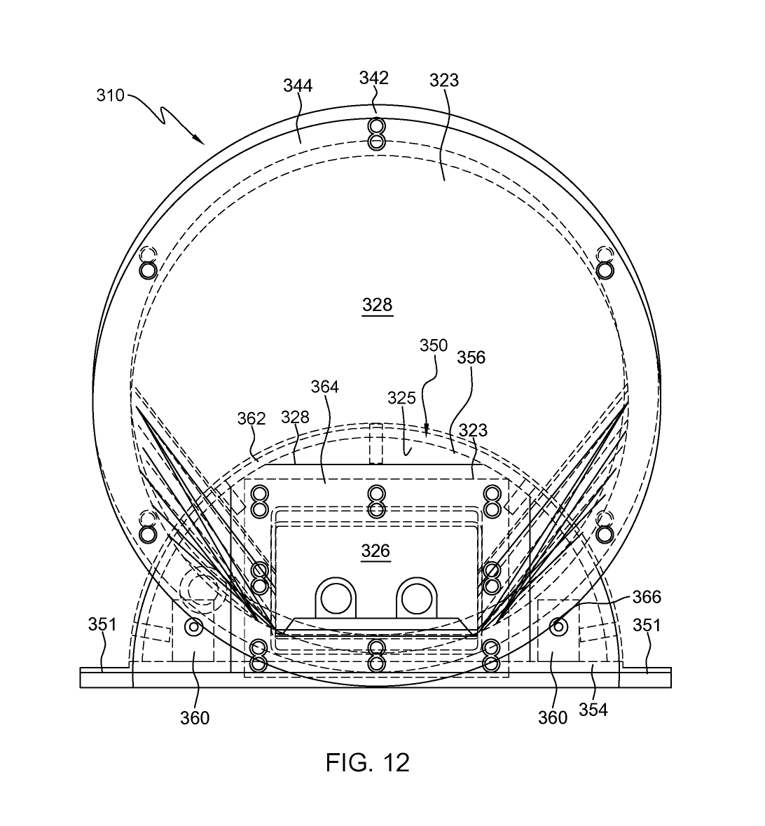

In FIGS. 7-12, an exemplary air treatment device, apparatus or system of the present disclosure is indicated generally by the reference numeral 310. The air treatment apparatus 310 is similar to components of the air treatment systems 10, 110 and 210 described above, and therefore like reference numerals preceded by the numeral “3” are used to indicate like elements, functions, aspects or the like. The air treatment apparatus 310 may be configured to be positioned between two portions of a plenum 312. For example, a portion of a plenum 312 of a transportation enclosure may be removed and the air treatment apparatus 310 may utilized to replace such a removed portion. As shown in FIGS. 7-12, the air treatment apparatus 310 includes an inlet portion 342 and an outlet portion 344. The inlet portion 342 and an outlet portion 344 may be configured to mate with corresponding portions of a plenum 312 such that the passage 328 of the air treatment apparatus 310 is in communication with the plenum 312. For example, as shown in FIGS. 7-12 the inlet portion 342 and outlet portion 344 may be circular at their outer edge or portion that is configured to mate with portions of a circular plenum 312. The inlet portion 342 and outlet portion 344 may also be substantially airtight. In this way, the inlet portion 341 may direct inlet air 314 flowing through the plenum 312 into and through the passageway 328 defined by the air treatment apparatus 310, and then return treated air 18 back to the plenum 312 via the outlet portion 344.

In some embodiments, the inlet portion 342 and/or outlet portion 344 may change cross-sectional size and/or shape (e.g., converge or diverge) as it extends between the plenum 312 and a treatment chamber 350 of the air treatment apparatus 310. The inlet portion 342 and/or outlet portion 344 may be removably coupled to the treatment chamber 350. In this way, the inlet portion 342 and/or outlet portion 344 may be provided in a numbering of differing shapes and/or sizes to mate with differing shaped and/or sized plenums 312, and coupled to the treatment chamber 350 so that the air treatment apparatus 310 can be utilized with any number of plenums 312.

The treatment chamber 350 of the air treatment apparatus 310 may be in communication with the inlet portion 342 and the outlet portion 344 such that the inlet portion 342 accepts and directs the inlet air 314 into the treatment chamber 350, the treatment chamber 350 treats the inlet air 314, and the outlet portion 344 returns the treated air 318 back to the plenum 312 (and/or into the transportation enclosure). The inlet portion 342, the treatment chamber 350, and the outlet portion 344 may thereby act in concert to form the passage 328 through the air treatment apparatus 310. The treatment chamber 350 may be substantially airtight such that the input air 314 fed or directed into the treatment chamber 350 via the inlet portion 342 is treated by the treatment chamber 350 (and output back to the plenum 312 via the outlet portion 344). For example, as shown in FIGS. 7-12 the air treatment apparatus 310 may include an inlet cap 362 and an outlet cap 364 coupled to the ends of the treatment chamber 350 to substantially close off the treatment chamber 350. The inlet portion 342 may be coupled to the inlet cap 362 and in communication with an aperture extending through the inlet cap 362. Similarly, the outlet portion 344 may be coupled to the outlet cap 364 and in communication with an aperture extending through the outlet cap 364. The inlet cap 362 and the outlet cap 364 may be coupled to the treatment chamber 350 in a substantially airtight manner such that the inlet cap 362 and the outlet cap 364 seal off the treatment chamber 350 but for the corresponding inlet portion 342 and outlet portion 344. In some embodiments, the outlet cap 364 may be fixed to the treatment chamber 350, while the inlet cap 362 may be removably or translatably coupled to the treatment chamber 350, as explained further below.

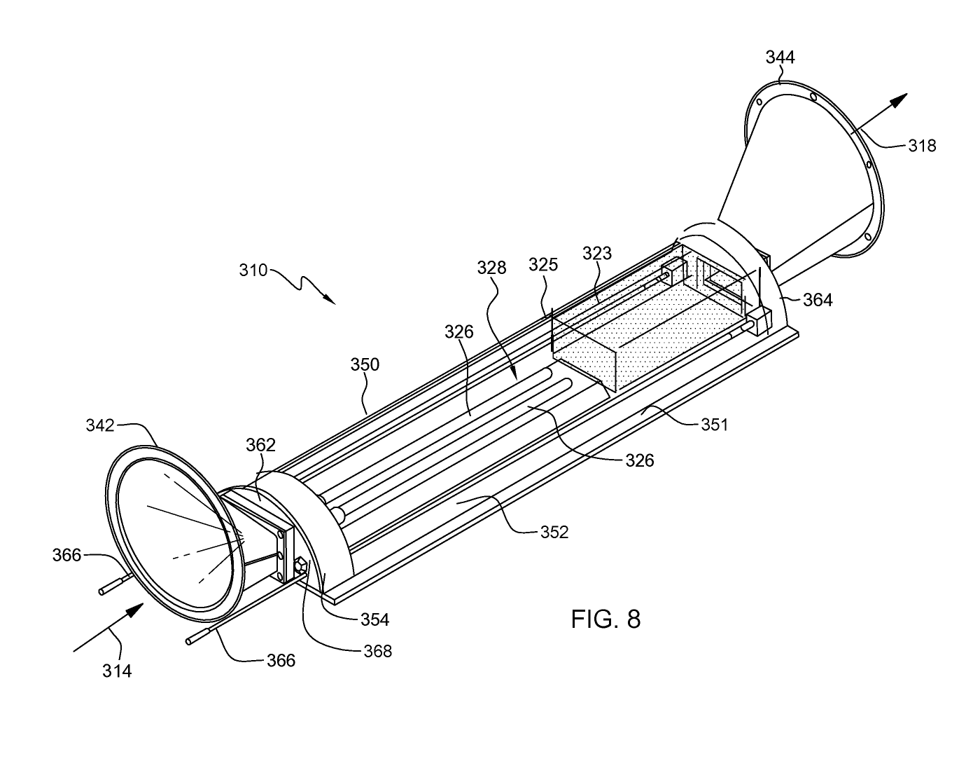

As shown in FIGS. 8-12, the treatment chamber 350 may form an elongate space or void between the inlet portion 342 and the outlet portion 344. The treatment chamber 350 may define a cross-sectional shape and size equal or lesser than the plenum 312, or the space in the transportation enclosure that houses the plenum 312. In this way, the treatment apparatus 310 may be provide in an existing space in a transportation enclosure that the plenum 312 is provided. As shown in FIGS. 7-12, the treatment chamber 350 may include a flange 351 extending from an outer surface or edge of the treatment chamber 350. The flange 351 may include one or more aperture and may be utilized, such as via fasteners, to affix or couple the treatment apparatus 310 to the transportation enclosure.

In the exemplary illustrated embodiment, the treatment chamber 350 is substantially dome- or semicircle shaped in cross-section, with the flange 351 extending outwardly from opposing sides of the planar portion of the semicircle shape. However, the treatment chamber 350 may be alternatively shaped. In some embodiments, the treatment chamber 350 may be extruded metal (e.g., a galvanized metallic duct member), or otherwise be formed or otherwise include a substantially rigid structure. The treatment chamber 350 may house or contain the at least one UV light emitting device 326 and the at least one downstream filter 323 (e.g., the second filter 324), and may include or define the reflective material 325 as shown in FIGS. 8-12.

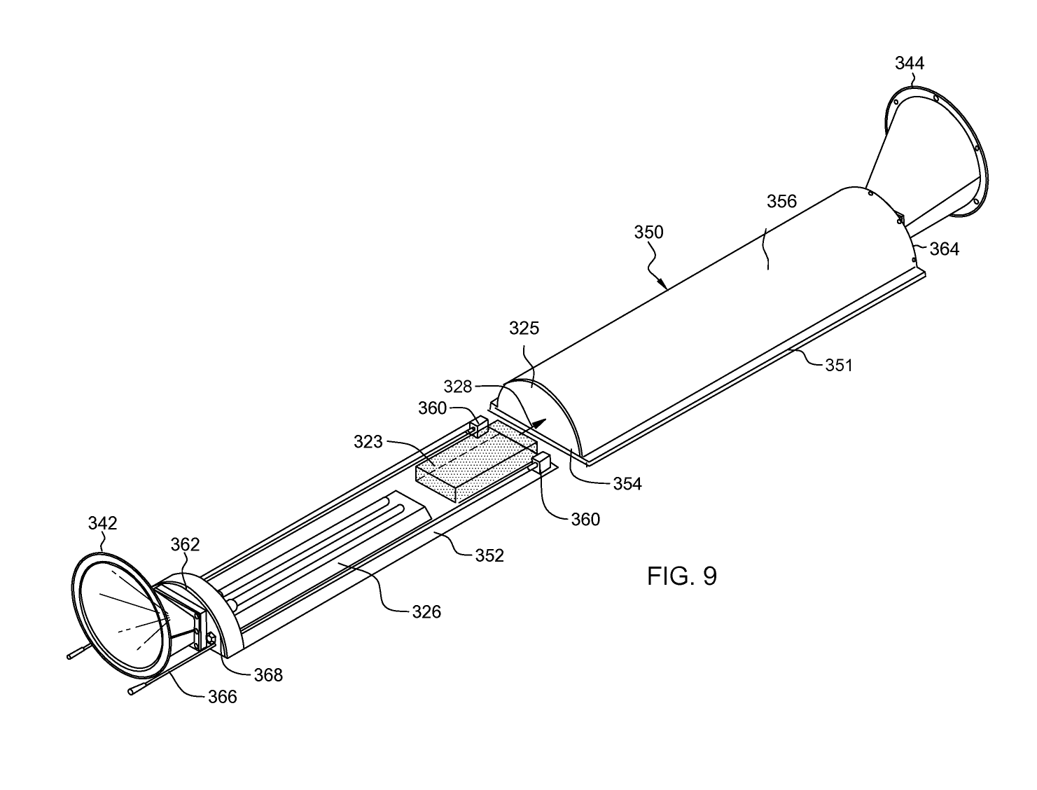

In some embodiments, the at least one UV light emitting device 326 and/or the at least one downstream filter 323 (e.g., the second filter 324) may be provided on a sled member 352 that is removably coupled within the treatment chamber 350, as shown in FIGS. 7-12. For example, the sled member 352 may be translatably coupled on a base portion 354 within the treatment chamber 350, as shown in FIG. 9. The sled member 352 may thereby slide or otherwise translate on the base portion 354 in/out of the treatment chamber 350. When positioned on the base portion 354 within the treatment chamber 350, the remainder of the interior of the treatment chamber 350 may extend about the sled member 352 (in cross-section). For example, as shown in FIGS. 8-12 the base portion 354 may be a substantially planar bottom portion of the semicircle-shaped treatment chamber 350, and the arced portion of the treatment chamber 350 may extend over the base portion 354. In this way, when the sled member 352 is positioned fully within the treatment chamber 350, the treatment chamber 350 may surround the sled member 352 (at least in cross-section) and at least the inner surface of the treatment chamber 350 other than the base portion 354 (on which the sled is positioned) may be exposed.

The inner surface of the treatment chamber 350 may include or define the reflective material 325. For example, the treatment chamber 350 may be formed of a metallic material that is polished or otherwise manufactured to define or provide the reflectivity properties noted above. In another embodiment, reflective material 325 may be coupled to the inner surface of the treatment chamber 350. In this way, the reflective material 325 of the inner surface of the treatment chamber 350 is configured to reflect the light emitted from the at least one UV light emitting device 326 when the sled member 352 is translated into the treatment chamber 350 (as discussed above).

As shown in FIGS. 7-12, the inlet cap 362 may be provided on, or affixed to, an end of the sled member 352 such that the inlet cap 362 and inlet portion 342 is removably or translatably coupled to the treatment chamber 350. Meanwhile, the outlet cap 364 may be fixed to the opposing end of the treatment chamber 350. As such, when the sled member 352 is positioned fully within the treatment chamber 350, such as on the base portion 354, the inlet cap 362 may seal or close off one end of the treatment chamber 350 but for the inlet portion 342, with the outlet cap 364 sealing or closing off the opposing end of the treatment chamber 350 but for the outlet portion 344.

The treatment apparatus 310 may be configured to prevent accidental or unnecessary exposure of UV light emitted from the at least one UV light emitting device 326 exterior to the treatment apparatus 310. For example, as shown in FIGS. 8-12 the treatment apparatus 310 may include one or more limit switch 360 or other mechanism that is configured to prevent activation of the at least one UV light emitting device 326 when the at least one UV light emitting device 326 is not positioned within the treatment chamber 350. The limit switch 360 may cut off power to the at least one UV light emitting device 326 unless the sled member 352 is positioned fully within the treatment chamber 350 with the inlet cap 362 sealing off the respective end of the treatment chamber 350. In this way, the limit switch 360 may only allow activation of the at least one UV light emitting device 326 when the at least one UV light emitting device 326 is sealed or closed off within the treatment chamber 350 to prevent accidental or unnecessary exposure of UV light to repairmen, installers, technicians, or passengers in the enclosure. As shown in FIGS. 8-12, wiring 366 for the one or more limit switch 360 (and/or the at least one UV light emitting device 326) may extend through the inlet cap 362 (or another portion of the treatment apparatus 310).

As the limit switch 360 or other mechanism may be configured to prevent activation of the at least one UV light emitting device 326 when the at least one UV light emitting device 326 is not positioned fully within the treatment chamber 350, the treatment apparatus 310 may be configured to provide a visual indication of whether or not the at least one UV light emitting device 326 is activated when it is positioned fully within the treatment chamber 350. As shown in FIGS. 7-12, the treatment apparatus 310 may include a window or visual port 366 that provides a visual sight line from exterior to the treatment apparatus 310 to within the treatment chamber 350. The visual port 366 may be substantially airtight. The visual port 366 may be utilized to visually check whether the at least one UV light emitting device 326 is or is not emitting UV light.

As shown in FIGS. 8-12, the treatment apparatus 310 may also contain the at least one downstream filter 323 (e.g., the second filter 324) within the treatment chamber 350. The at least one downstream filter 323 may be positioned between the at least one UV light emitting device 326 and the outlet portion 344, as described above. The at least one downstream filter 323 is configured within the treatment chamber 350 such that all the input air 315 must pass through the filter 323 before is passes through the aperture in the outlet cap 363 and the outlet portion 344 as treated air 318. For example, the at least one downstream filter 323 may tightly abut or otherwise block the aperture in the outlet cap 363. In some embodiments, the at least one downstream filter 323 may be positioned on the sled member 352, other otherwise arranged within the treatment chamber 350, such that when the sled member 352 is positioned fully within the treatment chamber 350 the at least one downstream filter 323 is positioned against the outlet cap 363 so that all input air 315 must flow through the at least one downstream filter 323.

As discussed above, the at least one downstream filter 323 may be positioned such that UV light emitted from the at least one UV light emitting device 326 irradiates, or acts upon, the at least one downstream filter 323. For example, the air treatment apparatus 310 may be configured such that UV-C light emitted from the at least one UV light emitting device 326 directly irradiates on the at least one downstream filter 323, and/or UV-C light emitted from the at least one UV light emitting device 326 indirectly irradiates on the at least one downstream filter 323 (e.g., at least one UV reflective material 325 of the treatment chamber 350 reflects UV-C light emitted from the at least one UV light emitting device 326 onto the at least one downstream filter 323). As also noted above, the treatment apparatus 310 may be configured such that at the flow rate of the input air 314, the UV light emitting device 326, the UV reflective material 325 and the at least one downstream filter 323 cooperate such that the output air 318 and any particles therein were subjected to a dose of UV light of at least 20 J/m² via only a single pass through the treatment apparatus 310. It is noted that such a dosage is effective to kill or inactivate a large percentage of microorganisms. It is also noted that any microorganisms trapped in the at least one downstream filter 323 will be subjected to a significantly larger dose of UV light. Further, in some embodiments the treatment apparatus 310 may be configured such the UV light emitting device 326 and the UV reflective material 325 cooperate such that the treatment apparatus 310 is at least a URV 13 UVGI system, and the at least one downstream filter 323 is at least a parameter 8 MERV ASHRAE Standard 52.2 filter. In some other embodiments, the treatment apparatus 310 may be configured such the UV light emitting device 326 and the UV reflective material 325 cooperate such that the treatment apparatus 310 is at least a URV 13 UVGI system, and the at least one downstream filter 323 is at least a parameter 13 MERV ASHRAE Standard 52.2 filter. Still further, in some other embodiments the treatment apparatus 310 may be configured such the UV light emitting device 326 and the UV reflective material 325 cooperate such that the treatment apparatus 310 is at least a URV 15 UVGI system, and the at least one downstream filter 323 is at least a parameter 15 MERV ASHRAE Standard 52.2 filter.

In some embodiments (not shown), the air treatment apparatus 310 may include a protective enclosure that acts as a moisture and/or temperature barrier to at least the at least one UV light emitting device 326. For example, the air treatment apparatus 310 may be wrapped or otherwise enclosed in a moisture and temperature barrier or insulator material. The protective enclosure may prevent or reduce the infiltration of moisture into the treatment chamber 350 and to the at least one UV light emitting device 326 above (or below) that of the input air 315. The protective enclosure may prevent or reduce thermal conductance (or the infiltration of warm/cold air) into the treatment chamber 350 and to the at least one UV light emitting device 326. For example, the protective enclosure may prevent or reduce the likelihood that the cold temperatures affect the operating temperature of the at least one UV light emitting device 326 below that of the input air 315.

The air treatment apparatus 310 may be utilized with any plenum 312 of any transportation enclosure. In some embodiments, multiple air treatment apparatuses 310 mat be utilized in a transportation enclosure, such as multiple air treatment apparatuses 310 along a particular plenum 312, or at least one air treatment apparatus 310 utilized within a plurality of differing plenums 312. In some embodiments, as discussed further below, the air treatment apparatus 310 may be a second or final stage treatment apparatus such that the prior treatment to the input air 314 has already been performed. Stated differently, the air treatment apparatus 310 may be utilized with another air treatment apparatus for a particular plenum 312, or may be utilized as the sole treatment for the air in the particular plenum 312. In some embodiments, the treated air 318 output by the air treatment apparatus 310 may be introduced into a transportation enclosure without further treatment.

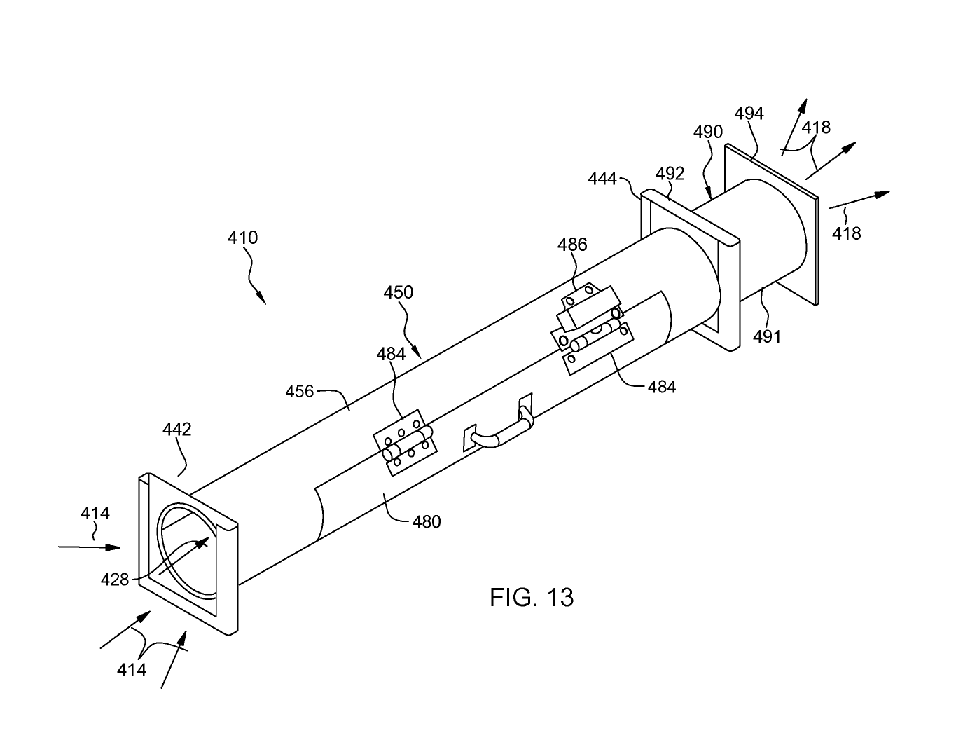

In FIGS. 13-16, an exemplary air treatment device, apparatus or system of the present disclosure is indicated generally by the reference numeral 410. The air treatment apparatus 410 is similar to components of the air treatment systems 10, 110, 210 and 310 described above, and therefore like reference numerals preceded by the numeral “4” are used to indicate like elements, functions, aspects or the like. The air treatment apparatus 410 may be configured to be positioned between two portions of a plenum 412. For example, a portion of a plenum 412 of a transportation enclosure may be removed and the air treatment apparatus 410 may utilized to replace such a removed portion. As shown in FIGS. 13-16, the air treatment apparatus 410 includes an inlet portion 442 and an outlet portion 444. The inlet portion 442 and an outlet portion 444 may be configured to mate with corresponding portions of a plenum 412 such that the passage 428 of the air treatment apparatus 410 is in communication with the plenum 412. For example, as shown in FIGS. 7-12 the inlet portion 442 and outlet portion 444 may include a flange at their outer edge or portion that is configured to mate with, potentially, a flange of a plenum 412. The inlet portion 441 may direct inlet air 414 flowing through the plenum 412 into and through the passageway 428 defined by the air treatment apparatus 410, and then return treated air 418 back to the plenum 412 via the outlet portion 444.

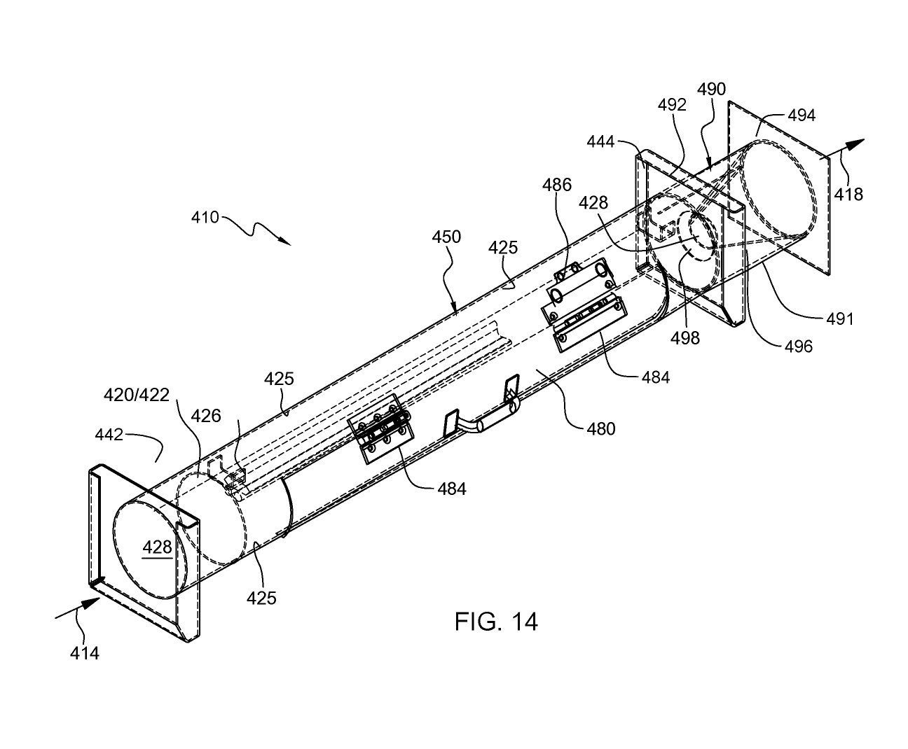

In some embodiments, the inlet portion 442 and/or outlet portion 444 may include a flange with at channel or groove that is configured to allow another flange or the like to mate or nest therein, as shown in FIGS. 13 and 14. In this way, a flange or other mating portion of a plenum 412 may be slid or otherwise moved into the channel of the inlet portion 442 and/or outlet portion 444 to couple the plenum 412 and the air treatment apparatus 410 and position the treatment chamber 450 in communication with the input air 414 flowing through the plenum 412. It is noted that the flange or other attachment member of the plenum may be a component or mechanism that is attached to an end of the plenum 412.

Similar to the air treatment apparatus 310 described above, the treatment chamber 450 of the air treatment apparatus 410 may accept and treat the inlet air 414, and return the treated air 418 back to the plenum 412 (and/or into the transportation enclosure). The treatment chamber 450 may thereby form the passage 428 through the air treatment apparatus 410. The treatment chamber 450 may be substantially airtight such that the input air 414 fed or directed into the treatment chamber 450 via the inlet portion 442 is treated by the treatment chamber 450 (and output back to the plenum 412 via the outlet portion 444). As shown in FIGS. 8-12, the treatment chamber 450 may form an elongate space or void between the inlet portion 442 and the outlet portion 444. The treatment chamber 450 may define a cross-sectional shape and size equal or lesser than the plenum 412, or the space in the transportation enclosure that houses the plenum 412. In this way, the treatment apparatus 410 may be provided in an existing space in a transportation enclosure in which the plenum 412 is provided.

In the exemplary illustrated embodiment, the treatment chamber 450 is substantially circular shaped in cross-section. However, the treatment chamber 450 may be alternatively shaped. In some embodiments, the treatment chamber 450 may be extruded metal (e.g., a galvanized metallic duct member), or otherwise be formed or otherwise include a substantially rigid structure. The treatment chamber 450 may house or contain the at least one UV light emitting device 426 and at least one of the first filter 420 and the third filter 422, and may include or define the reflective material 425 as shown in FIGS. 13-16.

In some embodiments, the at least one UV light emitting device 426 may be provided or coupled on or in a mounting bracket, or may otherwise be coupled within the treatment chamber 450. For example, the at least one UV light emitting device 426 may be attached to a bracket or other member within the treatment chamber 450 via fasteners. The inner surface of the treatment chamber 450 may include or define the reflective material 425. For example, the treatment chamber 450 may be formed of a metallic material that is polished or otherwise manufactured to define or provide the reflectivity properties noted above. In another embodiment, reflective material 425 may be coupled to the inner surface of the treatment chamber 450. In this way, the reflective material 425 of the inner surface of the treatment chamber 450 is configured to reflect the light emitted from the at least one UV light emitting device 426 (as discussed above).

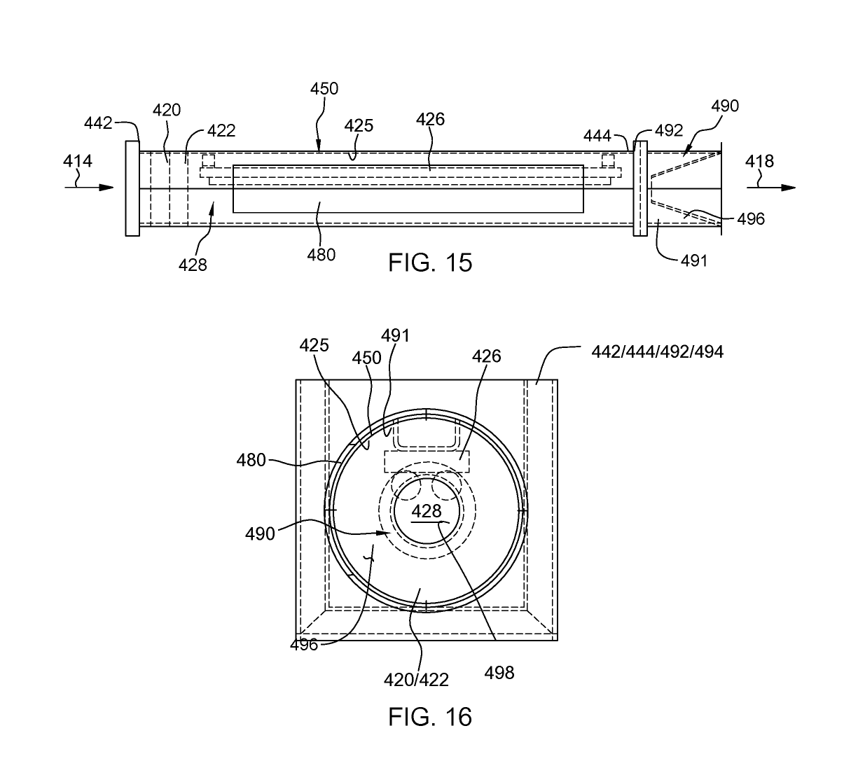

As shown in FIGS. 14 and 15, the treatment apparatus 410 may also contain the first filter 420 and/or the third filter 422 within the treatment chamber 450. The first filter 420 and/or the third filter 422 may be positioned between the at least one UV light emitting device 426 and the inlet portion 442 or the outlet portion 444. The first filter 420 and/or the third filter 422 may be configured within the treatment chamber 450 such that all the input air 415 must pass through the first filter 420 and/or the third filter 422 before is passes out the outlet portion 444 as treated air 418. For example, the first filter 420 and/or the third filter 422 may tightly abut or otherwise fill the passageway 428 (in cross-section) in the treatment chamber 450, such as proximate to the at the inlet or outlet portions 442, 444 and adjacent the at least one UV light emitting device 426. In some embodiments, the first filter 420 and/or the third filter 422 may be positioned within the treatment chamber 450 such that the first filter 420 and/or the third filter 422 abuts the UV reflective material 425 about the cross-section of the treatment chamber 450 and substantially entirely fills the passageway 428.

As discussed above, the first filter 420 and/or the third filter 422 may be positioned such that UV light emitted from the at least one UV light emitting device 426 irradiates, or acts upon, the first filter 420 and/or the third filter 422. For example, the air treatment apparatus 410 may be configured such that UV-C light emitted from the at least one UV light emitting device 426 directly irradiates on the first filter 420 and/or the third filter 422, and/or UV-C light emitted from the at least one UV light emitting device 426 indirectly irradiates on the first filter 420 and/or the third filter 422 (e.g., at least one UV reflective material 425 of the treatment chamber 450 reflects UV-C light emitted from the at least one UV light emitting device 426 onto the first filter 420 and/or the third filter 422). The treatment apparatus 410 may be configured such that at the flow rate of the input air 414, the UV light emitting device 426, the UV reflective material 425 and the first filter 420 and/or the third filter 422 cooperate such that the output air 418 and any microorganisms therein are subjected to a dose of UV light of at least 15 J/m² via only a single pass through the treatment apparatus 410. It is noted that such a dosage is effective to kill or inactivate a large percentage of microorganisms. It is also noted that any microorganisms trapped in the first filter 420 and/or the third filter 422 will be subjected to a significantly larger dose of UV light.

The treatment chamber 450 may include an access portion 480 that allows for ingress and egress within the treatment chamber 450, as shown in the 13-16. For example, as shown in FIGS. 13-16 the access portion 480 may be a separated portion of the treatment chamber 450 that is hinged 484 to a main portion of the treatment chamber 450. In some embodiments, treatment apparatus 410 may be configured to prevent accidental or unnecessary exposure of UV light emitted from the at least one UV light emitting device 426 exterior to the treatment. In some embodiments, the treatment apparatus 410 may include an interlock mechanism 486 that is operable to prevent the access portion 480 from opening or otherwise providing access to the treatment chamber 450, such as while the at least one UV light emitting device 426 is emitting UV light. The interlock mechanism 486 may alternatively be operable to prevent activation of the at least one UV light emitting device 426 when access portion 480 is open or otherwise provides access to the treatment chamber 450. For example, the interlock mechanism 486 may cut off power to the at least one UV light emitting device 426 unless the access portion 480 is closed or otherwise seals or closes off the treatment chamber 450. In this way, the interlock mechanism 486 may only allow activation of the at least one UV light emitting device 426 when the at least one UV light emitting device 426 is sealed or closed off within the treatment chamber 450 to prevent accidental or unnecessary exposure of UV light to repairmen, installers, technicians, or passengers in the enclosure. In some embodiments, the treatment apparatus 410 may include a window or visual port that provides a visual sight line from exterior to the treatment apparatus 410 to within the treatment chamber 450. The visual port may be utilized to visually check whether the at least one UV light emitting device 426 is or is not emitting UV light.

As shown in FIGS. 13-16, the treatment apparatus 410 may include at least one coupling 490 operable to couple or connect the treatment apparatus 410 to an end of a plenum 412. The coupling 490 is operable to couple the treatment apparatus 410 and a plenum 412 and account for any differences in shape and/or size therebetween. The coupling 490 may include a first end portion 492 and a second end portion 494 that are configured to mate with the treatment apparatus 410. For example, in the exemplary illustrated embodiment the first end portion 492 and the second end portion 494 may include a flange or other mechanism that is configured to mate within the groove or channel of the inlet portion 442 or outlet portion 444. In this way, the coupling 490 is operable to be installed at either the inlet portion 442 or outlet portion 444. It is noted that the coupling 490 may be utilized with any other apparatus or component other than the treatment apparatus 410 to connect or couple to an end of a plenum 412.

The coupling 490 may include an outer chamber 491 that defines a passageway therethrough and may be substantially airtight. The size and shape of the inner surface of the outer chamber 491 may substantially match that of the inner surface of the treatment chamber 450. When the coupling 490 and the treatment apparatus 410 are coupled or connected, the outer chamber 491 of the coupling 490 and the treatment chamber 450 may be in communication such that the passageway 428 extends through the chamber 491 of the coupling 490 and the treatment chamber 450. In this way, the coupling 490 may receive treated air 418 from the outlet portion 44 of the treatment chamber 450 of the treatment apparatus 410.

As shown in FIGS. 14 and 15, the coupling 490 may include an elastic, hollow tapered receiver member 496 within the chamber 491. The receiver member 496 may be coupled to the inner surface or side of the chamber 491 and extend along the length of the chamber 491 (i.e., along the direction of the flowing treated air 418) and taper inwardly as it extends from second end portion 494 to the first end portion 492. The tapered receiver member 496 may terminate at an aperture 498 before convergence however. As such, the passageway 428 extends through the aperture 498 and the diverging receiver member 496 along the direction of the flowing treated air 418.

The receiver member 496 may be made from any elastic material. The receiver member 496 may be operable to expand or deform outwardly (e.g., radially) when an end of a plenum 412 is translated into the receiver member 496 from the second end portion 494, and to exert a compressive force to the plenum 412 thereafter. As the receiver member 496 tapers inwardly along the direction extending from the second end portion 494 to the first end portion 492, the receiver member 496 may accept any plenum 412 configuration therein that is larger than the aperture 498 (and smaller than the size of the opening of the receiver member 496 proximate to the second end portion 494). Once an end of a plenum 412 is inserted into the opening of the receiver member 496 proximate to the second end portion 494 and forced into the receiver member 496, the receiver member 496 is able to expand and deform around the plenum 412 to create a substantially airtight seal therebetween. In this way, the receiver member 496 can accommodate any number of differing shaped and/or sized plenums 412 to couple a particular plenum 412 to the treatment apparatus 410 in a substantially airtight manner.

Substantially similar to the treatment apparatus 310, the treatment apparatus 410 may include a protective enclosure that acts as a moisture and/or temperature barrier to at least the at least one UV light emitting device 426. For example, the air treatment apparatus 410 may be wrapped or otherwise enclosed in a moisture and temperature barrier or insulator material. The protective enclosure may prevent or reduce the infiltration of moisture into the treatment chamber 450 and to the at least one UV light emitting device 426 above (or below) that of the input air 415. The protective enclosure may prevent or reduce thermal conductance (or the infiltration of warm/cold air) into the treatment chamber 450 and to the at least one UV light emitting device 426. For example, the protective enclosure may prevent or reduce the likelihood that the cold temperatures affect the operating temperature of the at least one UV light emitting device 426 below that of the input air 415.

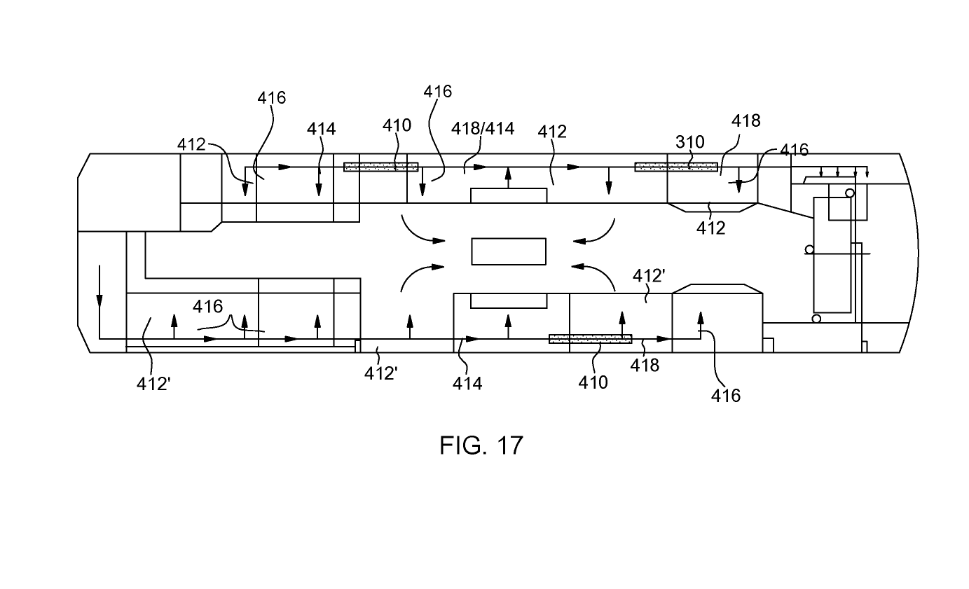

As shown in FIG. 17, the air treatment apparatus 410 may be utilized with any plenum 412 of any transportation enclosure, such as the illustrated exemplary motor vehicle. In some embodiments, multiple air treatment apparatuses 410 may be utilized in a transportation enclosure. For example, multiple air treatment apparatuses 410 along a particular plenum 412 may be utilized, or at least one air treatment apparatus 410 may be utilized within a plurality of differing plenums 412 of a transportation enclosure as shown in FIG. 17. In some embodiments, the air treatment apparatus 410 may be a first or pre-stage treatment apparatus such that the output treated air 418 is input air 414 to another treatment apparatus. For example, as shown in FIG. 17 a plenum 412 may include a first air treatment apparatus 410 to initially treat input air 414, and the output treated air 418 therefrom may be fed back into the plenum 412 and into the air treatment apparatuses 310 described above (as “input” air) as a second or final stage treatment apparatus. As shown in FIG. 17, after the input air 414 is treated by both the air first treatment apparatus 410 and the second treatment apparatus 310, the treated air 418 output by the second treatment apparatus 310 may be input 416 into the enclosure, such as proximate an operator's compartment or area. In contrast, as also shown in FIG. 17, another plenum 412′ of the enclosure may only include the air treatment apparatus 410 such that the output treated air 418 from the air treatment apparatus 410 is not treated further before it is input 416 into the enclosure.

It is to be understood that the above description is intended to be illustrative, and not restrictive. Numerous changes and modifications may be made herein by one of ordinary skill in the art without departing from the general spirit and scope of the invention as defined by the following claims and the equivalents thereof. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Also, the term “operably connected” is used herein to refer to both connections resulting from separate, distinct components being directly or indirectly coupled and components being integrally formed (i.e., monolithic). Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure. It is to be understood that not necessarily all such objects or advantages described above may be achieved in accordance with any particular embodiment. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the disclosure may include only some of the described embodiments. Accordingly, the invention is not to be seen as limited by the foregoing description, but is only limited by the scope of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An air treatment system for treating the air of a transportation enclosure that includes an air handling system with at least one plenum defining a flowpath of air through the air handling system and into the transportation enclosure via at least one input, the air treatment system comprising:
a plurality of filters positioned within the flowpath, at least two of the plurality of filters increasing in ASHRAE Standard 52.2 MERV parameters in the direction of the flowpath of air, wherein the filter positioned furthest downstream in the direction of flowpath of air is at least a parameter 8 MERV ASHRAE Standard 52.2 filter;
at least one ultraviolet (UV) light emitting device positioned within the flowpath; and
UV light reflective material positioned within the flowpath configured to increase the effective flux of the ultraviolet radiation of the at least one UV light emitting device within the flowpath,
wherein UV light emitted from the at least one UV light emitting device irradiates at least a portion of the filter positioned furthest downstream in the flowpath of air of the plurality of filters, and
wherein the plurality of filters, at least one UV light emitting device, and UV light reflective material are operable to provide a dose of UV light of at least 20 J/m² to the input air flowing through the air treatment system at a first volumetric flow rate.

2. The air treatment system of claim 1, wherein the first volumetric flow rate is less than about 700 cubic feet per minute.

3. The air treatment system of claim 1, wherein the first volumetric flow rate is less than about 550 cubic feet per minute.

4. The air treatment system of claim 1, wherein at least one UV light reflective material is at least 50% UV light reflective.

5. The air treatment system of claim 1, wherein at least the filter positioned furthest downstream in the direction of flowpath of air, the at least one UV light emitting device, and the at least one UV light reflective material are positioned within a treatment chamber, and wherein the treatment chamber is positioned between two portions of the at least one plenum such that the flowpath of air of the at least one plenum is directed to the treatment chamber and returned to the at least one plenum after flowing through the treatment chamber.

6. The air treatment system of claim 1, wherein the at least one UV light emitting device and the UV light reflective material are configured to provide a dose of UV light of at least about 30 J/m² to the input air flowing through the air treatment system.

7. The air treatment system of claim 1, wherein the plurality of filters includes at least three filters positioned along the flowpath of air.

8. The air treatment system of claim 7, wherein the filter positioned furthest upstream in the flowpath of air is positioned upstream of the at least one UV light emitting device.

9. The air treatment system of claim 8, wherein the filter positioned furthest upstream in the flowpath of air is at least a parameter 4 MERV ASHRAE Standard 52.2 filter.

10. The air treatment system of claim 9, wherein the filter positioned furthest downstream in the flowpath of air is at least a parameter 12 AS MERV ASHRAE Standard 52.2 filter, and wherein a third filter positioned between the furthest upstream filter and furthest downstream filter in the flowpath of air is at least a parameter 8 MERV ASHRAE Standard 52.2 filter.

11. The air treatment system of claim 1, wherein at least one of the plurality of filters, the at least one UV light emitting device, and at least one UV light reflective material are positioned within a first duct, and wherein the first duct is positioned within the at least one plenum.

12. The air treatment system of claim 11, wherein the flowpath of air of the at least one plenum is directed through the first duct.

13. The air treatment system of claim 1, wherein the filter positioned furthest downstream extends at least partially about the at least one UV light emitting device.

14. The air treatment system of claim 1, wherein UV light emitted from the at least one UV light emitting device is ultraviolet germicidal irradiation (UVGI) or short-wavelength ultraviolet radiation (UV-C).

15. The air treatment system of claim 1, wherein the total UV watt output of the at least one UV light emitting device is at least about 30 UV watts.

16. The air treatment system of claim 1, wherein at least one of the plurality of filters includes an anti-microbial coating.

17. The air treatment system of claim 1, wherein the enclosure is a railed or motor vehicle.

18. A transportation enclosure including the air treatment system of claim 1.

19. A method for treating air of a transportation enclosure that includes an air handling system with at least one plenum defining a flowpath of air through the air handling system and into the transportation enclosure via at least one input, the method comprising:
positioning a plurality of filters within the flowpath of air, at least two of the plurality of filters increasing in ASHRAE Standard 52.2 MERV parameters in the direction of the flowpath of air, wherein the filter positioned furthest downstream in the direction of flowpath of air is at least a parameter 8 MERV ASHRAE Standard 52.2 filter;
positioning at least one ultraviolet (UV) light emitting device within the flowpath of air and proximate at least the furthest downstream positioned filter of the plurality of filters in the direction of flowpath of air;
positioning UV light reflective material within the flowpath of air and about the at least one UV light emitting device to increase the effective flux of ultraviolet radiation emitted by the at least one UV light emitting device when energized within the flowpath of air; and
energizing the at least one UV light emitting device such that the at least one UV light emitting device irradiates at least a portion of the furthest downstream positioned filter and provides a dose of UV light of at least 15 J/m² to the flowpath of air flowing at a first volumetric flow rate.

20. An air treatment apparatus for treating the air of a transportation enclosure that includes an air handling system with at least one plenum defining a flowpath of air through the air handling system and into the transportation enclosure via at least one input, the air treatment apparatus comprising:
a hollow treatment chamber;
an input portion configured to couple to a first portion of the at least one plenum and direct the flow of air through the treatment chamber;
an output portion configured to couple to a second portion of the at least one plenum and direct the flow of air from the treatment chamber to the second portion of the at least one plenum;
at least a first filter positioned within the treatment chamber operable such that the flow of air through the treatment chamber flows through the first filter, the first filter being at least a parameter 12 MERV ASHRAE Standard 52.2 first filter;
at least one ultraviolet (UV) light emitting device positioned within the treatment chamber; and
UV light reflective material positioned within the treatment chamber configured to increase the effective flux of the ultraviolet radiation of the at least one UV light emitting device within the treatment chamber, and
wherein UV light emitted from the at least one UV light emitting device irradiates at least a portion of the first filter.
